(19) United States
(12) Patent Application Publication
(10) Pub. No.: US 2024/0033702 A1
(43) Pub. Date: Feb. 1, 2024

(54) METHOD FOR PREPARING BIODEGRADABLE MICROCASPULES AND USE OF THE RESULTING MICROCAPSULES

(71) Applicant: GEM INNOV, Gemenos (FR)

(72) Inventors: Yohann GUILLANEUF, Marseille (FR); Catherine LEFAY, Marseille (FR); Didier GIGMES, Allauch (FR); The Hien HO, Marseille (FR); Kaouthar OUDOUA, Marseille (FR); Thierry RIBEIRO, La Fare Les Oliviers (FR); Yves ORTAIS, Gemenos (FR); Marie-Pierre CESBRON, Gemenos (FR)

(73) Assignee: GEM INNOV, Gemenos (FR)

(21) Appl. No.: 18/253,068

(22) PCT Filed: Nov. 17, 2021
(86) PCT No.: PCT/IB2021/060653

§ 371 Date: May 16, 2023

(30) Foreign Application Priority Data
Nov. 17, 2020 (FR) 2011795
Nov. 19, 2020 (FR) 2011888

(51) Int. Cl. B01J 13/14; A01N 25/28; A01N 25/06; A61Q 15/00; A61K 8/11; A01N 65/12; A61K 8/85; A61K 9/50; A01P 7/04; C11D 11/00; C11D 3/00; C11D 17/00; C11D 3/50; C11D 3/37; C09B 67/02; D06M 23/12; D06M 15/356

(52) U.S. Cl. CPC B01J 13/14; A01N 25/28; A01N 25/06; A61Q 15/00; A61K 8/11; A01N 65/12; A61K 8/85; A61K 9/5031; A01P 7/04; C11D 11/0017; C11D 3/001; C11D 17/0039; C11D 3/505; C11D 3/3769; C09B 67/0097; D06M 23/12; D06M 15/3562; A61K 2800/412; D06M 2101/06

(57) ABSTRACT

Use of biodegradable microcapsules comprising a wall made of poly(beta-amino)ester, abbreviated to PBAE, which contains an active substance. These microcapsules may be obtained by a method of interfacial polymerization between an amine monomer and a multi-acrylate monomer. Depending on the intended use, microcapsules with a wall that is weak, breakable or unbreakable and porous or non-porous are used; these properties may be obtained by the choice of the monomers and the thickness of the wall. For certain uses, it is possible to use the polymerization reaction mixture directly, without washing.

### TECHNICAL FIELD OF THE INVENTION

The present description relates to the field of microcapsules, and more particularly to methods for manufacturing microcapsules with a view to enclosing active substances actives such as essential oils. More specifically, it relates to a method for preparing biodegradable microcapsules. This method performs interfacial polymerization of multifunctional compounds resulting in poly(beta-amino ester)s forming the shell of said microcapsules. The invention also relates to biodegradable microcapsules obtained with this method.

The present invention also relates to the use of the microcapsules obtained by the method according to the invention, depending on the mechanical and physicochemical properties of their shell.

### STATE OF THE ART

Microencapsulation is a method for protecting a reactive, sensitive or volatile substance (referred to here as “active ingredient”) in a capsule in which the size can vary from a nanometer to a micrometer. The core of the capsule is therefore isolated from the external environment thereof by a shell. This makes it possible to delay the evaporation, release or degradation thereof; there are numerous applications which make use of these technical effects when the microcapsules are incorporated in a complex formulation or applied to a product.

For example, microcapsules can be used to disperse in a controlled manner the active ingredient contained therein, which can particularly be a biocidal agent, an insecticide, a disinfectant, or a fragrance; this can take place by diffusion through the shell or under the influence of an external force which ruptures the shell. In some applications, the release of the active ingredient takes place under the influence of an external force which breaks the shell of the microcapsules; thus, it is possible to release an adhesive (see for example WO 03/016369-Henkel), or a reagent (see for example WO 2009/115671-Catalyse).

In further applications, the contents of the microcapsule cannot escape but the color change thereof under the effect of a variation of temperature (thermochromism) or of UV radiation (photochromism) is outwardly visible (see for example WO 2013/114 025-Gem Innov, or WO 2007/070118-Kimberly-Clark, or EP 1 084 860-The Pilot Ink Co.).

There are several techniques for preparing microcapsules. The main ones are spray-drying, interfacial polymerization, solvent evaporation, polymer self-assembly using the Layer-by-Layer (LbL) technique, and colloidosome preparation. All these techniques make it possible to obtain stable microcapsules of a mean diameter of 10 μm. Interfacial polymerization is nevertheless the predominant technique as it enables quick preparation in a single step of microcapsules in which the shell is strong enough for the latter to be isolated and thus be used in numerous applications.

The formation of microcapsules by interfacial polymerization is usually performed in 4 steps: (i) Preparing a first phase containing the active ingredient (for example an essential oil) and an organo-soluble monomer; (ii) Forming an emulsion by dispersing first phase in an aqueous medium containing surfactant, and which represents the second phase; (iii) Adding the water-soluble monomer to the second phase; (iv) Forming and maturing the membrane by reacting the monomers by polycondensation at the interface.

Several polymer families are conventionally used for manufacturing the shell of microcapsules (Perignon, C. et al., Journal of Microencapsulation 2015, 32 (1), 1-15), such as polyamides (PA), polyurethanes (PU) or polyureas. The preparation of PA microcapsule shells generally uses monomers of the diamine (hexamethylene diamine for example) and acyl chloride (sebacoyl chloride for example) type, whereas those in PU make use of monomers of the di-isocyanate (HDI, IPDI etc.) and diol type. In the case of polyureas, di-isocyanate and diamine type monomers or di-isocyanates alone are used wherein the hydrolysis at the interface produces amines enabling urea function synthesis.

For example, the document WO 2009/115671 cited above describes the formation of microcapsule shells by interfacial polycondensation, using different monomer mixtures:hexamethylene diisocyanate (HMDI) and ethylene diamine; tetraethylorthosilicate (TEO) and 3-(trimethoxysilyl)propylmethacrylate (MPTS); 2,4-tolylenediisocyanate (TDI) and 1,3 phenylenediamine; 2,4-toluene diisocyanate and 1,3-phenylene diamine.

There are already some works reporting the preparation of microcapsules by interfacial polymerization using other types of polymers. Mention can be made for example of the works by J. Bernard on the preparation of glyconanocapsules by copper-catalyzed azide-alkyne cycloaddition (R. Roux et al., J. ACS Macro Lett. 2012, 1 (8), 1074-1078), or the works by K.

Landfester (Siebert et al. Chem. Commun. 2012, 48, 5470-5472).

L. Shi et al. (J. Appl. Polym. Sci. 2016, 133 (36), 168-7) and D. Patton et al. (ACS Appl. Mater. Interfaces 2017, 9 (4), 3288-3293) who also prepared microcapsules by thiol-ene chemistry initiated by respectively a base and a photoinitiator.

A relatively broad spectrum of polymeric materials is therefore proposed to a person skilled in the art to select the suitable type of microcapsule for a given use. Thus, microcapsules are already used in numerous technical applications, but the application potential thereof has not yet been fully recognized, and it is a strongly emerging sector destined to grow significantly once the microcapsule shell meets increasingly stringent criteria in terms of toxicity and recyclability.

However, microcapsules represent microparticles of polymeric materials. For some years, polymeric material microparticles have been identified as an area of environmental concern, due to the wide dissemination thereof in ecosystems, in soils, in aquatic and maritime ecosystems, reaching distant locations from the place where they were introduced into the ecosystem.

This wide dissemination harms not only as a general rule the organisms present in these ecosystems, but could also have harmful effects for human health. Increasingly stringent regulations are already being announced which restrict the use of plastics capable of forming microparticles during the degradation thereof in-situ in a natural environment, and especially of plastics used directly in the form of microparticles.

For environmental reasons, it may seem contradictory to seek to develop a novel product consisting of polymeric microparticles. It has hence emerged as desirable to have microcapsules made of degradable polymeric material. It is noted that microcapsules, used in numerous special applications and capable of being incorporated in numerous products in common use (such as textile materials, cosmetic or phytosanitary products) or technical use (such as paints, varnishes, inks), will not normally undergo end-of-life collection, and therefore cannot undergo biodegradation by composting, as can be envisaged for collected plastic products. Thus, the degradability of the plastics forming the shell of microcapsules cannot be based on chemical mechanisms which take place during composting. In this context, the question as to whether the degradability of the microcapsules involves a biological mechanism is somewhat unimportant; what is important is the degradability thereof in an ecosystem, regardless of the chemical mechanism of this degradation. For example, a fermentation would be a biodegradation, while a simple degradation in an ecosystem under the effect of light could be a photochemical reaction independent of the ecosystem; in reality, the situation will often be a combination, especially if the degradation takes place in stages. We use the expression “(bio)degradable” hereinafter to denote the characteristic of a material of degrading in a natural environment on a relatively brief scale (of the order of weeks or a year), according to the characteristics of this natural environment and the exposure of the material to the various agents present in this natural environment.

It is observed that all the microcapsules previously developed result in the preparation of polymer chains (polyamide, polyurea, polyurethane, etc.) which will be either physically interlocked in the case of a reaction between bifunctional compounds, or crosslinked in the case of one or more multifunctional compounds (functionality Z 3). In any case, the shells are not (bio)degradable due to the nature of the polymer chain.

The problem addressed by the present invention is that of providing a novel type of microcapsules, which is easy to synthesize, without making use of toxic and/or costly raw materials, is (bio)degradable in the natural environment, can be used with a large number of active ingredients, and provides good external protection for the active ingredient that it is intended to contain. This new type of microcapsule allows a wide variety of new uses.

### OBJECTS OF THE INVENTION

During their research work, the inventors discovered that one possibility for obtaining degradable microcapsules would be to prepare shells made of polyester, which is a polymer known for the (bio)degradability thereof. The literature shows that studies have already been conducted on this theme, and it has been demonstrated that the rate of reaction between acid chlorides and diols was very slow. This system is thus unsuitable for interfacial polymerization (see E.M. Hodnett and D.A. Holmer, J Polym Sci, 1962, 58, 1415-21). Specific conditions such as the use of bisphenol A as diol and/or a reaction at very high pH made it possible to obtain microcapsules (see W. Eareckson, J Polym Sci, 1959, 399-406; see also P.W. Morgan and S.L. Kwolek, J Polym Sci, 1959, 299-327) but these conditions are overly restrictive for numerous internal phases and/or applications. Furthermore, the slow rate of polymerization reactions impedes the industrial use thereof in economic terms and in terms of short or even continuous production cycles.

Thus, the inventors did not pursue this avenue.

According to the invention, the problem is solved using microcapsules made of poly(beta-amino)ester (abbreviated here as PBAE). According to the invention, these microcapsules are synthesized in a single reaction step via an addition reaction of amine functions to acrylate functions (reaction known as “Michael addition”), by interfacial polymerization. This reaction results in the micro-encapsulation of the organic phase without forming by-products (see reaction diagram in FIG. 6). The presence of ester functions in the PBAE backbone gives the polymer good degradation properties via hydrolysis.

Poly(beta-amino ester)s are known per se and have been used substantially in recent years (Lynn, D. M.; Langer, R. J. Am. Chem. Soc. 2000, 122 (44), 10761-10768; Liu, Y.; Li, Y.; Keskin, D.; Shi, L. Adv. Healthcare Mater. 2018, 2 (2), 1801359-24) thanks to the biocompatibility and biodegradability properties thereof, and they now represent a family of materials which have numerous applications as biomaterials (for example as anticancer drug vector, as antimicrobial material, and for tissue engineering).

The areas of application of poly(beta-amino ester)s are very vast (see FIG. 9).

Generally, it is known that aza-Michael addition type reactions can be performed in a wide range of solvents ranging from halogenated nonpolar solvents (dichloromethane or chloroform for example) to polar solvents such as dimethyl sulfoxide (DMSO) for example (Liu, Y.; Li, Y.; Keskin, D.; Shi, L. Adv. Healthcare Mater. 2018, 2 (2), 1801359-24). In practice, the PBAEs are essentially prepared in solution and are subsequently formulated to produce for example micelles, particles, gel/hydrogels, or films (so-called Layer-by-Layer technique). Oligo-PBAEs have also been crosslinked in a second phase, either by photopolymerization (Brey, D. M.; Erickson, I.; Burdick, J. A. J. Biomed. Mater. Res. 2008, 85A (3), 731-741.7), or in the presence of di-isocyanates.

It is also known that linear or crosslinked PBAEs are relatively stable in neutral medium but are degraded more rapidly by ester function hydrolysis at acid and/or basic pH. This hydrolysis phenomenon results in the release of small molecules such as bis(β-amino acid)s and diols when linear PBAEs are used; these molecules are known to be non-toxic with respect to mammalian cells, and to have a weak influence on the metabolism of healthy cells.

According to an essential feature of the present invention, the microcapsules having a PBAE shell are synthesized by interfacial polymerization.

More specifically, according to the invention, the problem is solved by a method wherein the Michael polycondensation reaction between amine functions and acrylate functions is used to obtain Poly(Beta-Amino Esters) (PBAEs) by interfacial polymerization. The inventors discovered that this method, applicable to various active ingredients to be encapsulated, makes it possible to prepare stable microcapsules capable of being isolated by drying and which have the property of being (bio)degradable.

The microencapsulation method according to the invention comprises the following steps:
(a) Dispersion of one or more compounds having at least two acrylate functions in an organic solution (also referred to here as “oily phase”, in the context of an emulsion) forming the phase to be encapsulated (and comprising, where applicable, the active ingredient);
(b) Addition of an excess with respect to the preceding volume of an aqueous phase comprising one or more surfactants, followed by an emulsification;
(c) Addition to the emulsion obtained in step (b) of one or more compounds including at least one primary amine function and/or two secondary amine functions and polymerization reaction at a temperature between about 20° C. and 100° C.; to obtain microcapsules comprising a PBAE shell containing the phase to be encapsulated.

Depending on the intended use, this reaction mixture, referred to here as “primary reaction mixture”, can be used as it is, optionally after having diluted it with an appropriate liquid phase, or the microcapsules can be collected, washed and/or dried.

The new process for manufacturing microcapsules containing a so-called active substance can therefore be described as follows:
an aqueous surfactant solution, an oily phase comprising said active substance and at least a first monomer X, and a polar phase comprising at least one second monomer Y are provided;
an emulsion of O/W type is prepared by adding said oily phase to said aqueous surfactant solution;
said polar phase is added to said O/W emulsion, to enable a polymer to be obtained by polymerization of said monomers X and Y;
said process being characterized in that said polymer is a poly(beta-amino ester).

In an optional step, said microcapsules comprising a shell formed by said polymer and containing said active substance are isolated from this reaction mixture.

It is also possible, before or after this optional step, to modify the external surface of the shell of the microcapsules by a physical coating or by a chemical reaction.

Said first monomer X is selected from (multi)acrylates, particularly (multi)acrylates of formula X′—(—O(C═O)—CH═CH₂)n where n z 2 and where X′ is a molecule whereon n acrylate structural units are grafted.

Said first monomer X is, preferably, selected from (multi) acrylates of formula X′—(—O(C═O)—CH═CH₂)ₙ where n≥4 and where X′ is a molecule whereon n acrylate structural units are grafted. More specifically, it is advantageously selected from the group formed by:
diacrylates, and preferably those described in the article by Nayak et al. (Polymer-Plastics Technology and Engineering, 2018, 57, 7, 625-656);
triacrylates, particularly C₁₅O6H_{2O} (CAS No. 15625-89-5, i.e. trimethylolpropane triacrylate), tetraacrylates, pentaacrylates, hexaacrylates, mixtures of these different acrylates of type O[CH₂C(CH₂OR)₃]₂ where R is H or COCH═CH₂;
(multi)acrylates described in the article by Nayak et al. (Polymer-Plastics Technology and Engineering, 2018, 57, 7, 625-656);
polymers carrying pendant acrylate functions;
functional oligo-PBAEs, prepared for example by reacting diacrylate compounds with a functional primary amine and/or a functional secondary diamine;
the mixture of different compounds described above.

Said second monomer Y is selected from amines. More specifically, it is advantageously selected from the group formed by:
primary amines R—NH₂;
primary diamines of type NH₂(CH₂)nNH₂ where n is an integer which can typically be between 1 and 20, and which is preferably 2 or 6;
primary diamines comprising an aromatic core such as meta-xylylene diamine;
primary (multi)amines such as tris(2-aminoethyl)amine;
secondary diamines such as piperazine;
(multi)amines containing primary and secondary amine functions such as tetraethylene pentamine;
polymers containing primary and/or secondary amine functions such as polyethylene imine.

In an embodiment, said polymerization of said monomers is performed under stirring at a temperature between 20° C. and 100° C., and preferably between 30° C. and 90° C.

The microcapsule containing a so-called active substance, which can be manufactured by this method, is characterized in that its shell is formed of poly(beta-amino ester). This shell can be modified by adding a polymer layer deposited on the surface of the microcapsules. This deposition can be performed by adding a polymer dispersed in an aqueous phase which will be deposited on the surface of the capsules. Among these polymers, mention can be made of polysaccharides (for example, cellulose, starch, alginates, chitosan) and derivatives thereof.

Another possibility for modifying the shell of the microcapsules is that of modifying it by adding a radical initiator either in the aqueous phase or in the oily phase. A final possibility is that of reacting the residual surface amine functions with water-soluble monofunctional acrylates to modify the surface condition of the microcapsules.

A first object of the present invention is the use of microcapsules with a biodegradable shell made of Poly(Beta-Amino Ester), abbreviated PBAE, containing at least one active substance, in the formulation of products selected from the group formed by:
Cosmetic products and care products intended to be applied to the face or body, in particular body creams and lotions, face creams and lotions, shower gels, bath gels, products with UV filters, self-adaptive make-up products correcting their shade according to ambient solar or electric lighting, said at least one microencapsulated active substance being in particular intended to be released in a controlled and targeted manner, in particular for vectorization in the skin, for stabilizing pigments and/or dyes, for covering wrinkles.
Hair and scalp care products, including shampoos, conditioners, hair dyes.
Depilatory waxes, said at least one microencapsulated active substance being preferably selected from the group formed by thermochromic substances and agents for temperature control.
Disinfectant and/or deodorant products intended to be applied to the face or body, or to objects in contact with the body, to control or limit or mask body odor or to control or limit or mask the odor of objects in contact with the body, said at least one active substance being preferably selected from the group formed by antiperspirants, perfumes, essential oils, fragrances, oils.
Cosmetic textiles, intended to come into contact with the skin, said at least one active substance preferably being selected from the group formed by antioxidant products, anti-cellulite products, products capable of stimulating blood circulation, anti-mold products, antimicrobial products, bactericidal products, virucidal products, oils, colorants, UV screens, surfactants, vitamins, moisturizing agents, skin penetration enhancers, exfoliating agents, emollients, anti-wrinkle agents, depigmentation agents, humectants; knowing that this group of products can also apply to any other product for cosmetic use and to any composition for cosmetic use.
Medical textiles, such as dressings, intended to come into contact with the skin or with wounds, said active substance being a pharmaceutical active ingredient.
Pharmaceutical preparations, in particular intended for use orally, nasally, intravenously, rectally or by topical application, for example cutaneous or ocular, said at least one active substance preferably being selected from the pharmaceutical active ingredients.
Preparations for the care of the teeth and the mouth, said at least one active substance being preferably selected from bactericidal products, enzymes, flavorings, essential oils, sweeteners.
Food preparations, said at least one active substance being preferably selected from flavorings, flavor enhancers, vitamins and vitamin preparations, preservatives, nutritional additives, oils.
Insecticidal and/or fungicidal and/or repellent and/or anti-moss preparations, intended for use alone or incorporated into products such as phytosanitary products, paints, coatings, textiles, detergents, plastics.
Preparations intended for use in the field of agriculture, said at least one active substance being preferably selected from additives for the cultivation of plants and fertilizers.
Preparations intended for veterinary, intracorporeal or extracorporeal, use, and in particular pharmaceutical preparations, dermatological preparations and coat care preparations, said at least one active substance being preferably selected from pharmaceutical active ingredients and in particular dewormers and antiparasitics, food additives, perfumes, fragrances.
Preparations for the treatment of textile products, textile fibers or footwear, said at least one active substance preferably being selected from biocidal products, repellents, moth repellents, deodorizing products, self-cleaning products, stain-resistant products, antistatic products, self-healing products, self-repairing products, perfumes, fragrances, detergents, softeners.
Preparations for the treatment of bedding, in particular mattresses, pillows, sheets, undersheets, luggage, fabrics and household equipment such as curtains, cushions, furniture, leather objects, said at least one active substance being preference selected from among biocidal products, repellents, moth repellents, anti-mite products, anti-lice products, anti-mosquito products, deodorant products, self-cleaning products, antistatic products, self-healing products, self-repairing products, perfumes, fragrances, detergents, softeners.
Preparations for waterproof or water-repellent coatings, intended to be applied in particular to textile surfaces or leather surfaces, said at least one active substance preferably being selected from hydrophobic compounds, in particular fatty compounds or fluorinated compounds.
Deodorant and/or disinfectant and/or antifoam and/or antifungal preparations, said at least one active substance preferably being selected from oily, natural or synthetic products.
Detergent products.
Preparations for coating building materials, preparations for ensuring watertightness and preparations for thermal insulation or for absorbing and restoring thermal energy, said at least one active substance preferably being selected from phase change products, paints and varnishes, thermal insulation materials, sealing agents, expansion agents.
Products for treating floor coverings, said at least one active substance being preferably selected from the group formed by lubricant products, anti-friction products, anti-slip products, detergents, essential oils, fragrances, perfumes, aromas.
Paints, varnishes, dyes and inks, in particular inks for transfer printing, screen printing, ink jet printing or electrostatic printing, and preparations for coating or making paper, said at least one active substance preferably being selected from the group formed by dyes, pigments, thermochromic products, perfumes, aromas, fragrances.
Self-repairing compositions intended in particular for use in surface coatings used in paint, varnish, inks, on cement, concrete, wood, and in compositions of polymer materials and composites.
Compositions with a fire retardant and fire extinguisher effect, said at least one active substance being able in particular to be chosen from brominated alkanes of general formula CnH₂n+₂₋ₓBrₓ.

Said microcapsules which form the basis of this new use are obtainable by a method in which:
(a) an aqueous surfactant solution, an oily phase comprising said active substance and at least a first monomer X, and a polar phase comprising at least a second monomer Y are provided;
(b) an O/W type emulsion is prepared by adding said oily phase to said aqueous surfactant solution;
(c) said polar phase is added to said O/W emulsion, in order to obtain a polymer by polymerizing said monomers X and Y, said polymer forming the shell of said microcapsules; and in which said first monomer X is preferably selected from (multi)acrylates, and preferably (multi)acrylates of formula X′—(—O(C═O)—CH═CH₂)n with n>4 and where X′ represents a molecule on which n acrylate units are grafted, or selected from the group formed by:
diacrylates;
triacrylates, in particular trimethylolpropane triacrylate, tetraacrylates, pentaacrylates, hexaacrylates, mixtures of these different acrylates of the O[CH₂C(CH₂OR)₃]2 type where R is H or COCH═CH₂;
polymers bearing pendant acrylate functions;
functional oligo PBAEs, prepared for example by reacting diacrylate compounds with a functional primary amine and/or a functional secondary diamine;
the mixture of different compounds described above.

In one embodiment, said second monomer Y is selected from amines, and preferably selected from the group formed by:
primary amines R—NH₂;
primary diamines of type NH₂(CH₂)nNH₂ where n is an integer which can typically be between 1 and 20, and which is preferably 2 or 6;
primar diamines comprising an aromatic core, and preferably meta-xylylene diamine;
primary (multi)amines such as tris(2-aminoethyl)amine;
(multi)amines containing primary and secondary amine functions such as tetraethylene pentamine;
secondary diamines, and preferably piperazine;
polymers containing primary and/or secondary amine functions, and preferably polyethylene imine.

The ratio of the reactive functions of said monomers Y(—NH functions) and X (acrylate functions) is greater than 1, preferably between 1 and 5, and even more preferably between 1.2 and 3.8. It should be noted that for a —NH₂ group, two—NH functions are counted here.

Said polymerization of said monomers takes place under stirring at a temperature of between 20° C. and 100° C., and preferably between 30° C. and 90° C.

The outer shell of the microcapsules can be modified by one of the following ways:
Deposition of a coating on the surface of the microcapsule, preferably from a polymer dispersed in an aqueous phase, said polymer preferably being selected from polysaccharides, such as cellulose, starch, alginates, chitosan, and their derivatives.
Addition of a radical initiator in the aqueous phase and/or the oily phase, said radical initiator preferably being selected:
When added to the aqueous phase: in the group formed by water-soluble azo compounds, such as 2,2′-Azobis(2-methylpropionamidine) dihydrochloride, and red-ox systems, such as ammonium persulfate or potassium in combination with potassium metabisulphite;
When added to the oily phase: in the group formed by azo compounds, such as azobis-isobutyronitrile and its derivatives, and peroxidic compounds, such as lauroyl peroxide;
Addition to the aqueous phase of a water-soluble acrylate capable of modifying the surface state of the microcapsules, said acrylate preferably being a water-soluble monofunctional acrylate capable of reacting with the residual amine functions at the surface of the shell of the microcapsules, said water-soluble monofunctional acrylates being preferably selected from the group formed by 2-carboxyethyl acrylate, 2-(dimethylamino)ethyl acrylate, 2-hydroxyethyl acrylate, poly(ethylene glycol) acrylates, the potassium salt of 3-sulfopropyl acrylate.

More generally, it can also be modified so as to have an anionic or cationic character.

Said microcapsules are characterized by one of the following characteristics or combination of characteristics:
an indentation force at break greater than 5,000 pN, preferably greater than 6,000 pN, and even more preferably greater than 7,000 pN; these microcapsules being referred to herein as “unbreakable”;
an indentation force at break of less than 300 pN and preferably a Tg value of less than 33° C., and in this case this Tg value being preferably less than 32° C. and even more preferably less than 31° C.; preferably by an indentation force at break of less than 250 pN and preferably by a Tg value of less than 32° C., and in this case this Tg value being preferably less than 31° C. and even more preferably less than 30° C.; and even more preferably by an indentation force at break of less than 200 pN and preferably by a Tg value of less than 32° C., and in this case this Tg value being preferably less than 31° C. and even more preferably below 30° C.; these microcapsules being referred to herein as “fragile”;
an indentation force at break between 200 pN and 5,000 pN, and preferably between 300 pN and 5,000 pN, but with a breaking force between 200 pN and 300 pN their Tg value is preferably at least 33° C., and for the microcapsules with a breaking force greater than 300 pN, it is preferable that their Tg value be between 30° C., preferably between 33° C. and 80° C.; these microcapsules being referred to herein as “brittle”.

For some uses it is preferred to use fragile microcapsules, for others brittle microcapsules, for still others unbreakable microcapsules. Brittle or unbreakable microcapsules can be more or less porous (or permeable), depending on the nature of the monomers chosen and the thickness of the shell; this porosity or permeability can be exploited for some of the new uses which will be described below, and for others it must be avoided.

According to an embodiment which is advantageous for certain uses, the reaction mixture resulting from step (c) can be used directly, optionally after adjustment of the pH value (which can be useful in particular in the event of an excess of amine). A slurry is thus used which can enter directly into liquid, viscous or pasty preparations, without having washed, isolated and/or dried the microcapsules beforehand. This simplifies the manufacturing process.

According to another embodiment which is advantageous for certain uses, the reaction mixture resulting from said reaction for modifying the external surface of the microcapsules can be used directly.

For still other uses it is preferable to use the microcapsules after washing and possibly drying. Such microcapsules with a purified surface are particularly suitable for uses in pharmacy and cosmetics.

A second object of the invention is a method of using microcapsules with a biodegradable shell made of Poly(Beta-Amino Ester), containing at least one active substance, in the formulation of products in the group listed above in relation to the first object of the invention. This method of use may include a step in which the microcapsules, preferably in a slurry form, are incorporated into a liquid, viscous or pasty preparation. This preparation can then be used as it is (for example in the form of an ink, a cosmetic or phytosanitary product) so as to allow the microcapsules to fulfill a desired technical function, and/or it can represent a vehicle for applying the microcapsules on another product, in particular on a solid product, for example on a textile fabric, to deploy there a desired technical function (for example a disinfectant effect for a textile).

A third object of the invention is a product capable of being obtained by the method of using microcapsules with a biodegradable shell made of Poly(Beta-Amino Ester), containing at least one active substance, said product being selected from the group listed above in relation to the first object of the invention.

### FIGURES

FIGS. 1 to 23 illustrate certain aspects of the invention, but do not limit their scope. FIGS. 2 to 5 relate to Example 1. FIG. 7 relates to Example 2, FIG. 8 to Example 3, FIG. 8 to Example 3, FIG. 10 to Example 6, FIG. 11 to Example 7, FIG. 12 to example 10, FIG. 13 to example 11, FIG. 14 to example 13, FIG. 15 to example 14, FIG. 16 to example 15, FIG. 17 to example 17, FIG. 18 to example 18, FIG. 21 to example 26, FIG. 22 to example 27, and FIG. 23 to example 28. FIGS. 2 to 5 and 10 to 14 are optical micrographs; the horizontal bar at the bottom left of the image represents a length of 50 μm. FIGS. 17 and 18 are also optical micrographs. FIGS. 21 to 23 are thermogravimetric analysis (TGA) curves.

FIG. 1 shows the general diagram of the method according to the invention. The four-digit reference numbers denote steps of this method.

FIG. 2 shows an optical micrograph of microcapsules obtained according to example 1, after 5 hours of reaction.

FIG. 3 shows a Fourier transform infrared (FTIR) spectrum of the shell of the microcapsules isolated in slurries after 6 hours of reaction.

FIG. 4 shows an optical micrograph of microcapsules obtained according to example 1, after drying on a glass strip.

FIG. 5 shows two optical micrographs of microcapsules obtained according to example 1, after drying on a glass strip. The left micrograph was obtained in grazing incidence light, the right micrograph under fluorescent light after adding some drops of a fluorescent dye.

FIG. 6 illustrates the reaction diagram of the reaction according to the invention.

FIG. 7 shows that the thermochromic microcapsules are stable after a 30-min oven passage and that the thermochromic function thereof is preserved.

FIG. 8 illustrates the degradability of the shells of the microcapsule with an accelerated degradation test.

FIG. 9 illustrates the different fields of application of poly(beta-amino ester)s.

FIG. 10 shows that the microcapsules are stable after 24 hours, and the mean diameter thereof is between 10 μm and 30 μm.

FIG. 11 shows a similar image to FIG. 10, and leads to the same conclusion, for another example.

FIG. 12 shows the result of the use of the microcapsules according to the invention in a carbonless copy paper (self-copying paper).

FIG. 13 shows a photograph of microcapsules according to another example of the invention.

FIG. 14 shows a photograph of microcapsules according to another example of the invention.

FIG. 15 shows the biodegradation percentage as a function of time for dry microcapsules according to the invention.

FIG. 16 shows the biodegradation percentage as a function of time for the shell of the microcapsules according to the invention.

FIG. 17 shows a photograph of microcapsules according to another example of the invention.

FIG. 18 shows a photograph of a cotton fiber which has been placed in contact with microcapsules according to the invention in which the surface has been modified (b) or not (a).

FIG. 19 shows the chemical formula of an amine carrying an anionic charge, which can be used as a monomer.

FIG. 20 shows the chemical formula of a polyacrylate, which can be used as a monomer.

FIG. 21 shows the thermogravimetric analysis curves of three samples according to an example of the invention, namely unmodified microcapsules containing the active phase (curve (b)), microcapsules surface-modified with potassium persulfate (curve (c)), and of the active phase alone (essential oil of eucalyptus, curve (a)).

FIG. 22 shows curves similar to those of FIG. 21, curve (c) relating to microcapsules surface-modified by the initiator azobisisobutyronitrile.

FIG. 23 shows curves similar to those of FIG. 22, curve (c) relating to microcapsules modified by photochemical polymerization.

### DETAILED DESCRIPTION

In the following detailed description of embodiments of the present description, numerous specific details are disclosed in order to provide a more in-depth understanding of the present invention, and to enable a person skilled in the art to execute the invention. However, it will be obvious to a person skilled in the art that the present description can be implemented without these specific details. In other cases, well-known features have not been described in detail to avoid overburdening the description unnecessarily.

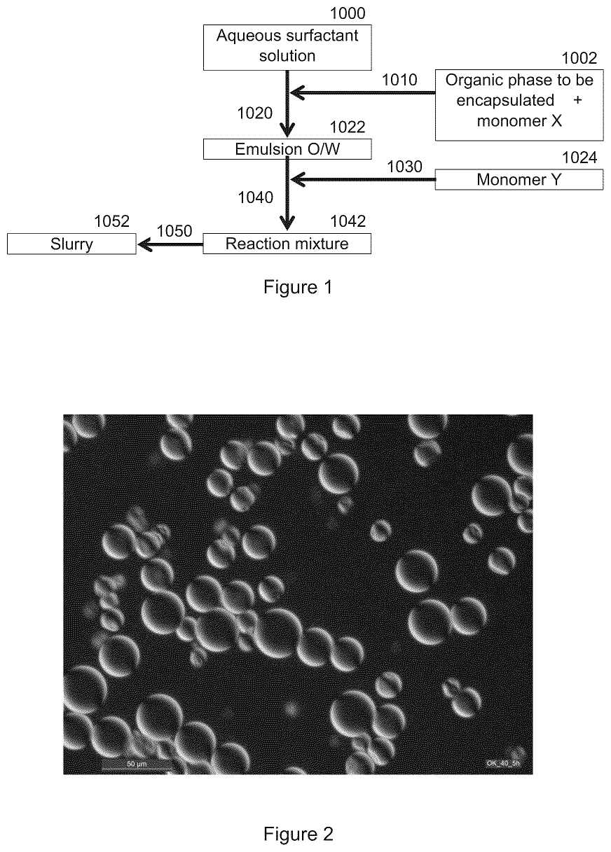

FIG. 1 shows a general diagram of the method according to the invention. The aqueous surfactant solution (1000) is prepared. An organic solution (also known as “oily phase”) is also prepared comprising the phase to be encapsulated (which comprises the so-called active substance) and the monomer X (1002). At step 1010, this oily phase 1002, which is an organic solution, is added to said aqueous solution 1000 and at step 1020 an O/W (oil-in-water, according to a term known to a person skilled in the art) type emulsion 1022 is obtained. In this emulsion, said organic solution is the so-called oily phase (0 phase). At step 1030, an aqueous solution of the monomer Y 1024 is added to said emulsion 1022. At step 1040, the polymerization reaction results in a reaction mixture 1042 from which, at step 1050, a heterogenous mixture 1052 known as slurry is formed, which comprises, suspended in an aqueous base, the microcapsules containing the phase to be encapsulated. It may contain a residue of unreacted monomers; this will be the case in particular if an excess of amine is used. This heterogeneous mixture (slurry) is called in the context of the present invention the “primary reaction mixture”. It can be used as it is, or be the subject of various operations of washing and/or collection and/or drying of the microcapsules contained in this heterogeneous mixture.

Step 1050 involves as a general rule a temperature of the reaction mixture 1042 greater than about 20° C., typically between 20° C. and 100° C. A temperature between about 30° C. and about 90° C. is preferred, and even more preferably between about 40° C. and about 80° C.

This method can be applied to different monomers X and Y.

According to the invention, the monomer X is a (multi)acrylate, and the monomer Y is an amine, preferably, a primary amine and/or a primary (multi)amine and/or a secondary diamine and/or a compound having primary and secondary amines.

The term (multi)acrylate denotes any compound of formula X′—(—O(C═O)—CH═CH₂)n where n≥2 and where X′ is a molecule whereon n acrylate structural units are grafted.

The term primary (multi)amine denotes any compound comprising at least two primary amine functions.

As an acrylate (monomer X), it is possible to use for example triacrylates (such as C₁₅O6H₂O, CAS No. 15625-89-5); tetraacrylates; pentaacrylates; hexaacrylates; mixtures of these different acrylates cited. It is possible to use for example molecules of type O[CH₂C(CH₂OR)₃]₂ where R can be H or COCH═CH₂.

It is possible to use PBAE oligomers, this term here includes functional PBAE oligomers and prepolymers, the chain ends of which are functions of the acrylate type; they can be prepared, for example, by reaction of diacrylate compounds with a functional primary amine and/or a functional secondary diamine.

As an amine (monomer Y), it is possible to use for example molecules of type NH₂(CH₂)nNH₂ where n is an integer which can typically be between 1 and 20, and which be for example 2 (ethylene diamine) or 6 (hexamethylene diamine, CAS number: 124-09-4). It is also possible to use piperazine, meta-xylylene diamine, pentaethylenehexamine, tris(2-aminoethyl)amine (TREN) or polyethylene imine (PEI).

Amines having an aliphatic carbon skeleton (for example hexamethylene diamine) having many degrees of freedom generally increase the deformation at break of the shell of the microcapsules obtained.

To reduce the deformation at break of the shell, it is possible to use more rigid multi-amines, for example meta-xylylene diamine.

The nature and the concentration of the amines and the acrylates can be varied.

The reagent function ratio of the monomers Y(—NH) and X (acrylate) is advantageously greater than 1, and typically between 1 and 5, preferably between 1.2 and 3.8.

According to a specific embodiment of the invention, the monomers X (acrylate) and/or Y (amine) are biosourced.

FIG. 6 shows the reaction diagram of the aza-Michael addition reaction between a secondary amine and an acrylate (reaction (a)) and the polyaddition reaction between a multifunctional acrylate compound and a multi-amine compound resulting in a cross-linked polymer (reaction (b)).

The organic core of the microcapsules can consist of an organic phase comprising an active substance. During the formation of the microcapsule, this organic (oily) phase will be enclosed by the polymeric shell of the microcapsule, which protects it from the environment. Said organic (oily) phase can consist of said active substance, or said active substance can be part of said organic (oily) phase, wherein it can be particularly dissolved. The expression “active substance” refers here to the specific purpose wherein the microcapsules are intended to be used; as a general rule, in view of the specificity of the microcapsule product, this purpose is always known during the manufacture thereof.

The active substance can be selected particularly from oils (pure or containing possibly other molecules in solution or in dispersion), such as essential oils, natural and edible oils, plant and edible oils, liquid alkanes, esters and fatty acids, or from dyes, inks, paints, thermochromic and/or photochromic substances, fragrances, products with biocidal effect, products with fungicidal effect, products with antiviral effect, products with phytosanitary effect, pharmaceutical active ingredients, products with cosmetic effect, adhesives; these active ingredients being optionally in the presence of an organic vector.

It is possible to use, non-restrictively, distillation extracts of natural products such as essential oils of eucalyptus, citronella, lavender, mint, cinnamon, camphor, aniseed, lemon, orange, which can be obtained by extraction from plant matter, or by synthesis.

It is also possible to use other substances such as long-chain alkanes (for example tetradecane), which can contain lipophilic solutions in solution.

As a general rule, and according to the function sought for the microcapsules, it is possible to use any hydrophobic compound, which will thus be naturally dispersed in the form of emulsion of hydrophobic droplets suspended in an aqueous phase.

Numerous additives enabling superior protection of the organic (oily) phase to be encapsulated, against infrared radiation, ultraviolet radiation, unintentional entry of specific gas or oxidation, can be incorporated in the microcapsule.

As surfactant, it is possible in particular to use nonionic surfactants, such as polyvinyl pyrrolidone (PVP), polyethylene glycol sorbitan monopalmitate (known under the trade mark Tween 20¹), polyethylene glycol sorbitan monolaurate (known under the trade mark Tween 40¹), polyethylene glycol sorbitan monooleate (known under the trademark Tween 80^{T}h), or ionic surfactants, such as partially neutralized salts of polyacrylic acids such as sodium or potassium polyacrylate or polymethacrylate, or lignosulfate sodium. It is possible to use copolymers of acrylic acid-alkyl acrylate, polyacrylic acid, polyoxyalkylene fatty esters. Use can be made of those nonionic surfactants which are mentioned in the Encyclopedia of Chemical Technology, volume 8, pages 912 to 915, and which have a lipophilic hydrophilic balance (according to the HLB system) greater than or equal to 10.

Other macromolecular surfactants can also be used. Mention can be made for example of polyacrylates, methylcelluloses, carboxymethylcelluloses, polyvinyl alcohol (PVA) optionally partially esterified or etherified, polyacrylamide or synthetic polymers that have anhydride or carboxylic acid functions such as ethylene/maleic anhydride copolymers.

Preferably, polyvinyl alcohol can be used as a surfactant.

It can be necessary, for example in the case of aqueous solutions of a cellulosic compound, to add a small amount of alkaline hydroxide such as soda, in order to facilitate the dissolution thereof; it is also possible to directly use such cellulosic compounds in the form of the sodium salts thereof for example. Amphiphilic copolymers of the Pluronics type can also be used. Generally aqueous solutions containing from 0.1 to 5% by weight of surfactant are used.

The size of the droplets is according to the nature and the concentration of the surfactant and the stirring speed, with the latter being chosen all the more so large as the desire to obtain smaller average diameters of droplets.

In general the stirring speed during the preparation of the emulsion is from 5,000 to 10,000 revolutions per minute. The emulsion is usually prepared at a temperature comprised between 15° C. and 95° C.

Generally when the emulsion has been obtained, stirring by turbine is stopped and the emulsion is stirred using a slower stirrer of the current type, for example of the frame stirrer type, typically at a speed of about 1,500 to 1,500 revolutions per minute.

The method according to the invention thus leads to homogeneous and fluid suspensions containing, according to the charges introduced, generally from 20% to 80% by weight of microcapsules having from 100 nm to 100 μm in average diameter. The microcapsules are spherical. The diameter of the microcapsules can preferably be comprised between 1 μm and 50 μm, and even more preferably between 10 μm and 40 μm. The mean diameter of the microcapsules can be determined using laser diffraction (Mastersizer™ type device), a known technique and device for this use.

Depending on the monomers, and in particular the amines, used for the polymerization, the external surface of the shell of the microcapsules can be of an anionic nature or of a cationic nature. An inherent anionic capsule can be obtained when using in aqueous phase amines an amine carrying an anionic charge; such an amine is shown in FIG. 19. An inherent cationic capsule can be obtained when an amine carrying a cationic function is used; an example is the partial quaternization of multi-amines in the aqueous phase.

The shell of the microcapsules can be modified by applying a surface coating. The deposition of said coating can be carried out by adding a polymer dispersed in an aqueous phase which will deposit onto the surface of the capsules. Among the polymers that can be used to this end, polysaccharides (cellulose, starch, alginates, chitosan) and their derivatives can be mentioned. This addition can be made at elevated temperature or at room temperature at the end of the interfacial polymerization.

The shell of the microcapsules can also be modified by adding a radical initiator either to the aqueous phase or in the organic phase (oil phase). The addition to the organic phase can be carried out before and/or after the preparation of the PBAE shell. When the radical initiators are added after the preparation of the shell, the radical initiator can be diluted in acetone in order to favour its penetration into the microcapsules. Said initiators can be azoic compounds (such as azobis-isobutyronitril and its derivatives) or peroxidic compounds (such as lauroyl peroxyde). When the radical initiators are added to the aqueous phase, the initiators can in particular be water soluble azoic compounds (such as 2,2′-Azobis(2-methylpropionamidine)dihydrochloride) or redox systems (such as ammonium or potassium persulfate in combination with potassium metabisulfite). Under inert atmosphere the radicals generated by the decomposition of the radical initiators can react with the residual acrylate functions in the PBAE shell and thereby increase its mechanical strength and/or modify its polarity.

Another way to modify the shell of the microcapsules is to make react their residual amine functions on the surface with water soluble monofunctional acrylates. While the inventors do not wish to be bound by this theory, they believe that through a Michael addition, amino-ester bondings are formed which can fix a functional group onto the surface. Among the water soluble acrylates that can be used, the following examples can be mentioned: acrylic acid, 2-carboxyethyle acrylate, 2-(dimethylamino)ethyl acrylate, 2-hydroxyethyle acrylate, poly(ethylene glycol) acrylates, the potassium salt of 3-sulfopropyl acrylate.

These various possibilities of modifying the shell of the microcapsules after their formation make it possible in particular to confer on them in a targeted manner anionic or cationic properties; this choice will essentially depend on the intended use for these microcapsules.

Here we describe methods to render the shell of microcapsules anionic.

A first method is post-modification by the radical route, using suitable aqueous azo compounds. To this end, the compound 4,4′-Azobis(4-cyanovaleric acid) (CAS No: 2638-94-0), available under the reference V501 from FujiFilm Wako, can be used.

A second method is post-modification by the radical route, using redox systems such as APS (ammonium persulphate, CAS no. 7727-54-0) or KPS (potassium persulphate, CAS no. 7727-21-1) and reducing agents of the iron or metabisulphite type.

A third method is the post-modification by Michael reaction between the secondary and primary amines still present on the surface of the capsules and anionic water-soluble acrylates of the acrylic acid type.

A fourth method is to apply a coating to the microcapsules with anionic polymers such as PAA or PMMA.

Here we describe post-modification methods to render the shell of microcapsules cationic.

A first method is the quaternization of the amines (which are mainly tertiary) present on the surface of the capsules by reaction with an alkyl halide in the aqueous phase.

A second method is post-modification by the radical route, using suitable aqueous azo compounds. To this end, the compound 2,2′-Azobis(2-methylpropion amidine)dihydrochloride (CAS No: 2997-92-4), available under the reference V50 from Fuji Film Wako, can be used.

A third method is the post-modification by Michael reaction between the secondary and primary amines still present on the surface of the capsules and water-soluble acrylates. DMAEA may be suitable as a water-soluble acrylate.

The microcapsules according to the invention can be prepared with very different mechanical properties; these mechanical properties can be tailored to the intended use, as will be explained in greater detail below. They are represented in the context of the present invention by the indentation force at break of the microcapsule, measured during a nanoindentation test at room temperature with a pyramidal point of the Berkovich type, and by the glass transition temperature determined with common differential thermal analysis techniques.

This indentation force at break depends on the intrinsic mechanical characteristics of the shell material, as well as the thickness of the shell.

In the context of the present invention, three types of microcapsules are distinguished, which are designated here by the adjectives “fragile”, “brittle” and “unbreakable”.

The so-called fragile microcapsules are characterized by an indentation force at break of less than 300 pN and preferably by a Tg value of less than 33° C., and in this case this Tg value is preferably less than 32° C. and even more preferably below 31° C. They are preferably characterized by an indentation force at break of less than 250 pN and preferably by a Tg value of less than 32° C., and in this case this Tg value is preferably less than 31° C. and even more preferably below 30° C. They are characterized even more preferably by an indentation force at break of less than 200 pN and preferably by a Tg value of less than 32° C., and in this case this Tg value is preferably less than 31° C. and even more preferably below 30° C.

These microcapsules can have a volume ratio of encapsulated substance relative to the shell (this ratio is known as “core/shell ratio”) which can reach 95/5 or even 96/4. In other words, the thinness of their shell contributes to their fragility.

Fragile microcapsules are used when rapid release of the active substance they contain is desired. This release can be facilitated by a temperature above room temperature. By way of example, such microcapsules can be used in compositions for the treatment of the hair and the scalp, which are exposed to a temperature above room temperature and to a very low mechanical pressure during their application. Another example of the use of active substances that can be enclosed in fragile microcapsules are the pigments or dyes used in cosmetic preparations.

The so-called brittle microcapsules are characterized by an indentation force at break of between 200 pN and 5,000 pN, and preferably between 300 pN and 5,000 pN. Microcapsules with a breaking force between 200 pN and 300% N preferably exhibit a Tg value of at least 33° C. For the microcapsules with a breaking force greater than 300 pN, it is preferable that their Tg value be between 30° C. (preferably between 33° C.) and 80° C.

These brittle microcapsules therefore have a low elongation at break, which leads to the fragmentation of the shell into pieces under a mechanical stress of sufficient force, for example when rubbing a surface which has been coated with these brittle microcapsules.

These microcapsules can be obtained with so-called rigid multi-amine monomers, such as meta-xylylene diamine; these compounds increase the Tg value of the shell.

Another possibility to lead to brittle microcapsules is to increase the crosslinking density of the shell to limit its elastic deformation. Mention may be made in this respect of pentaethylene hexamine which leads to a large quantity of knots.

Brittle microcapsules can be used for applications in which it is desired to be able to cause the release of the active substance by the application of mechanical pressure or by a chemical modification of the microcapsule which modifies its mechanical properties. An example is the use of microencapsulated active substances in blowing agents and sealants.

The shell of the brittle microcapsules may exhibit porosity.

In general, the shell of a microcapsule represents a diffusion barrier for the chemical species contained by the shell of the microcapsule; this is one of the goals of micro-encapsulation.

However, depending on the use envisaged for the microcapsules, it may be desirable to have a certain permeability of the shell to the molecules it contains. This permeability of the shell is also called its “porosity”, because at the atomic level, the permeability of the shell is not homogeneous over the entire surface of the shell: within the polymer there are more permeable zones than others, which are called “pores”.

In general, it is therefore desirable to be able to adapt the permeability of the shell to the intended use of the microcapsule. It is known that the permeability of a microcapsule shell is both determined by the nature and structure of its material, and it decreases when the thickness of the shell increases.

More specifically, for microcapsules having a PBAE shell, the porosity of the shell depends on the thickness of the shell and its chemical characteristics. The main chemical characteristic is the degree of cross-linking of the shell: a low degree of cross-linking generally results in greater porosity. For example, the knot density is low when the POSS™-acrylate monomer is used compared to an acrylate of the dipentaerythritol penta-/hexa-acrylate type: there are around fifteen carbon atoms between the knots instead of from five to seven: the POSS makes it possible to increase the rigidity of the shells and at the same time leads to a certain porosity.

The use of a multi-acrylate like the one shown in FIG. 20 gives a shell as rigid as that obtained with POSS, but the network is denser and the porosity lower than with POSS. The modulation of the porosity can also be done by mixtures of multi-acrylate compounds with a combination of short compounds of the diacrylate and multi-acrylate types (for example dipentaerythritol penta-/hexa-acrylate). This makes it possible to ensure that the majority of the acrylate functions have reacted because the short compound is more mobile and makes it possible to finish the crosslinking.

The porosity characteristic of the shell is irrelevant for fragile-type microcapsules, which are bound to release their contents rapidly by breaking the shell.

The so-called unbreakable microcapsules are characterized by an indentation force at break greater than 5,000 μN, preferably greater than 6,000 μN, and even more preferably greater than 7,000 μN. They preferably have a Tg value greater than 80° C., preferably a Tg value greater than 85° C., and are even more preferably a Tg value greater than 90° C.

According to a first variant, they have an indentation force at break greater than 5,000 μN and a Tg value greater than 80° C., preferably greater than 85° C., and even more preferably greater than 90° C.

According to a second variant, they have an indentation force at break greater than 6,000 μN and a Tg value greater than 80° C., preferably greater than 85° C., and even more preferably greater than 90° C.

According to a third variant, they have an indentation force at break greater than 7,000 μN and a Tg value greater than 80° C., preferably greater than 90° C., and even more preferably greater than 100° C.

It should be noted that the “unbreakable” nature of these microcapsules can result from two different and even contradictory characteristics: either the microcapsules are so rigid that they cannot be broken with the indentation force applied, or they are so elastic that they deform under the applied pressure, but without breaking.

It is also possible to vary the porosity of the unbreakable microcapsules, by the choice of appropriate monomers as described in relation to the brittle microcapsules. For certain uses, the so-called unbreakable microcapsules must not have significant porosity. The absence of porosity allows them to be used in applications where it is not desired that the active substance can leave the microcapsule, either by rupture of the shell or by passage through the shell. It is thus possible to encapsulate thermochromic substances or photochromic substances, intended to fulfill this function for a long period. Another use for which total hermeticity of the shell of the microcapsule is required is that of encapsulated phase change materials.

For other uses, for example for the diffusion of perfumes over a long period, the shell must be unbreakable, to avoid a too rapid release of the perfume, but must all the same allow the perfume to cross the shell: one will choose for that a shell of unbreakable type and porous type.

The increase in the mechanical properties of the shell can be obtained by introducing into the shell organic or inorganic groups which will serve as reinforcement for the shell. For example, the monomer POSS™-acrylate, comprising a siloxane cage modified by eight acrylate functions, gives the shell of the microcapsule an increase in Young's modulus. It is the same for the acrylate whose structure is shown in FIG. 20.

The glass transition temperature Tg of the microcapsules can be determined according to techniques known to those skilled in the art, with commercially available devices, for example a TGA 8000 type thermogravimetry device manufactured by the company PerkinElmer. For this measurement, the microcapsules are emptied and dried at room temperature, then a sample of the order of 10 mg to 50 mg (typically 25 mg) is transferred into the measuring cup. It is heated under nitrogen with a heating rate of about 20° C./min under a flow of nitrogen (20 ml/min). This measurement can also be carried out on a sheet of polymer obtained under similar reaction conditions. The porosity of the shells can be determined with the same device on the filled microcapsules: a sample of the same quantity is maintained at a fixed temperature (for example 70° C.) under a flow of nitrogen for approximately 17 h to 20 h, and the mass loss is recorded.

The microcapsules, and in particular their shell, according to the invention are (bio)degradable. Biodegradation can be determined for example by one of the methods described in the document “OECD Guidelines for the Test/s of Chemicals: Ready Biodegradability” (adopted by the Council of the OECD on Jul. 17, 1992). In particular, the manometric respirometry test (301 F method) can be used. Preferably, this test is carried out on emptied and washed microcapsules, so that the biodegradation of the contents of the microcapsules does not interfere with the test, the purpose of which is to characterize the biodegradation of the material forming the shell of the microcapsules. Preferably, the microcapsule according to the invention, and/or its shell, show a biodegradation of at least 80%, preferably of at least 83%, more preferably of at least 85%, measured after an incubation of 10 days using said 301 F method. With this same method, after incubation for 28 days, the microcapsules according to the invention preferably show a biodegradation of at least 90%, preferably of at least 95%, and even more preferably at least 98%.

The microcapsules according to the invention can be the subject of numerous uses. In general, they can be used in different ways.

The primary reaction mixture can be used as it is, optionally after having adjusted its pH. It is also possible to modify the surface of the microcapsules, as described above; in this case, the reaction mixture of this modification reaction (this reaction mixture being referred to here as “secondary reaction mixture”) can be used as it is (optionally after adjustment of the pH).

In all cases, dry microcapsules can be used, with or without washing (in the latter case, the reaction mixture is dehydrated).

The microcapsules are used dry especially in two cases: when it is necessary to integrate them in a hydrophobic medium (for example in a hydrophobic plastic material, such as a silicone), and when it is desired to observe a color change through the shell of the microcapsule; this will be the case when the microcapsule contains a thermochromic or photochromic product.

The use of washed microcapsules is generally necessary for pharmaceutical applications, for food applications, and for most cosmetic applications, because the reaction mixtures may contain molecules that are toxic, allergenic or otherwise incompatible with the intended use, or generally undesirable.

The microcapsules according to the invention have many uses which will be described below. For all the uses for which it is indicated that the reaction mixture can be used directly, this refers to the so-called primary reaction mixture as it results from the succession of the synthesis steps (a), (b) and (c) described above, optionally after adjustment of the final pH; this also refers to the so-called secondary reaction mixture which results from a reaction aimed at giving the outer shell of the microcapsule anionic or cationic properties. It is also possible to use a slurry prepared by purification of the reaction mixture (primary or secondary), or a slurry prepared from isolated microcapsules, possibly after washing and/or drying of the microcapsules, or a preparation comprising a mixture of the reaction mixture (primary or secondary) or one of the slurries which have just been described, optionally after addition of solvents or other functional constituents. Direct use of a reaction mixture is the simplest use, but purification or isolation or drying of the microcapsules, which require additional process steps, may have advantages.

For all the uses according to the invention, it is possible to use microcapsules according to the invention of a single type (composition and shell thickness), comprising one or more encapsulated active substances, or several types of microcapsules according to the invention (for example of the same shell composition but of different shell thickness, to have different release levels, or else of different shell composition) comprising one or more active substances, in the same microcapsule or in separate microcapsules.

The microcapsules can be used in cosmetic products and care products intended to be applied to the face and to the body, in particular to stabilize and/or vectorize one or more specific active substances and to control their release. Said cosmetic products and care products may in particular be creams and lotions for the body, creams and lotions for the face, shower gels, bath gels, UV filter products, more particularly self-adaptive make-up correcting their tint depending on ambient solar or electric lighting and other products comprising micro-encapsulated active substances intended to be released in a controlled and targeted manner, in particular for vectorization in the skin, for stabilization of pigments and/or dyes, for covering of wrinkles. Said cosmetic products and care products intended to be applied to the body may contain as active substances microencapsulated catalysts and/or enzymes, and/or microencapsulated wrinkle recoverers.

They may also be depilatory waxes, comprising thermochromic agents and/or agents for temperature control and/or active products with controlled release, said agents and active products being microencapsulated according to the invention.

It can also be products for hair and scalp care, and in particular shampoo, conditioners, hair dyes. They may also be disinfectant and/or deodorant products and products to control the smell of objects in contact with the body to control or limit or mask body odor; as such, the microencapsulated active products can in particular be antiperspirants, perfumes, essential oils and/or fragrances.

To use the microcapsules according to the invention in cosmetic products and care products intended to be applied to the face or body, it is generally preferred not to use the reaction mixture directly, but to wash, and preferably to isolate the microcapsules; in one embodiment, the microcapsules are isolated and dried before incorporating them into a cosmetic or care product preparation. For use in cosmetic products and care products, it is preferred to use microcapsules of the fragile, brittle or porous type, as the case may be; they are preferably anionic, but can also be cationic (in particular for shower gel and bath products, and for hair care products). For shower gel and bath products, the use of dry microcapsules or unbreakable type microcapsules does not present a significant advantage. For use in deodorants and for odor control the use of fragile or unbreakable type microcapsules is not preferred.

To incorporate the microcapsules into a depilatory wax preparation, the reaction mixture can also be used directly, and/or these microcapsules are preferably of the unbreakable and anionic types.

For most uses for cosmetic products, microcapsules of the anionic type, washed and/or isolated, possibly dried, are preferred. To incorporate microcapsules containing substances capable of filtering out UV radiation in cosmetic products, microcapsules of the fragile or unbreakable type, preferably anionic, are advantageously used. To encapsulate the pigments or dyes for use in these cosmetic compositions, microcapsules of the fragile, brittle or unbreakable type, preferably anionic, are advantageously used. To encapsulate other dermatological or cosmetic active ingredients, microcapsules of the porous or brittle type, preferably anionic, are used.

The microcapsules can be used to coat cosmetic textiles. For these uses, the microcapsules may contain antioxidant products, anti-cellulite products, products capable of stimulating blood circulation, as well as anti-mold, antimicrobial, bactericidal and virucidal products. For most of these uses, the reaction mixture can be used directly, although this is not preferred. The microcapsules can be of the porous type; for antioxidant and anti-cellulite products as well as for antimicrobial, bactericidal and virucidal products, it is also possible to use microcapsules of the brittle type. For all these uses, the microcapsules can be of the anionic or cationic type.

The microcapsules may contain pharmaceutical active ingredients and may be incorporated into pharmaceutical preparations which are intended to be used, in human or veterinary medicine, in particular orally, nasally, intravenously, rectally, or by topical application, for example cutaneous or ocular. These uses can in particular serve to release in a targeted manner the content of the microcapsules, which is typically a pharmaceutical active ingredient, in a specific chemical environment, this environment being for example characterized by a specific pH value. For all these pharmaceutical preparations washed microcapsules are used.

They are typically of the brittle type, and preferably of the porous type; they can, depending on the case, be of the anionic or cationic type.

To coat medical textiles, such as dressings, intended to come into contact with the skin or with wounds, the same type of microcapsule shells are used as for cosmetic textiles, but after washing; these microcapsules contain pharmaceutical active ingredients.

Said pharmaceutical active ingredients can in particular be hormones, corticosteroids, antiseptics, anti-oestrogens, antifungals, antibiotics, vasoactive agents, antiglaucoma agents, beta-blocking agents, cholinergic agents, sympathomimetic agents, inhibitors of carbonic anhydrase, mydriatic agents, virostatic agents, antitumor agents, antiallergic agents, vitamins, anti-inflammatory agents, immunosuppressive agents, anesthetic agents, ciclosporin, antioxidants, peptides, proteins, enzymes, polyphenols, and combinations and/or mixtures of these agents.

The microcapsules may contain adhesives and/or glues and may be used in the preparation of formulation of adhesives and/or glues. Preferably, the reaction mixture is used directly, without washing. Preferably, microcapsules of the brittle type, of the anionic or cationic type are used.

The microcapsules may contain photochromic agents, in particular for hair dyes and for protection products against sunlight. For hair care products, the reaction mixture is preferably used without washing, for products for protection against sunlight, washed microcapsules are preferably used.

For these two applications, it is possible to use microcapsules of the fragile type or of the unbreakable type, and preferably of the anionic type.

Reversible photochromic agents, which have many applications such as anti-counterfeiting agents, for recreational applications, writing and detection of UV radiation can also be contained in cationic type microcapsules.

The microcapsules may contain erasable or non-erasable inks, possibly scented, intended in particular for writing accessories and toys. These microcapsules can be of the brittle type or of the unbreakable type, and preferably of the anionic type. The reaction mixture can be used without washing.

The microcapsules may contain detergent products, in particular for wet cleaning or for dry and/or waterless cleaning. These microcapsules can be of the brittle type and/or of the porous type. They can be of the anionic or cationic type. The reaction mixture can be used without washing, and/or dried microcapsules can be used.

The microcapsules can be used in products for the care of the teeth and the mouth, where they can contain specific active products. These microcapsules are preferably used after washing, possibly after drying. The microcapsules can be of the fragile type, of the brittle type, of the unbreakable type, and/or of the porous type. They may contain active deodorizing substances.

The microcapsules can be used in food preparations. In this use, they contain active food products, such as flavorings, flavor enhancers, vitamins, nutritional additives, preservatives. Washed, preferably isolated, optionally dried microcapsules are used. They can be fragile, brittle or porous. They may be of the anionic type.

The microcapsules can be used to turn liquid oil into powder.

One can use these microcapsules containing the liquid oil after washing and drying to incorporate them into cosmetic or food preparations. For this, microcapsules of the brittle type, which are preferably of the anionic type, are preferred.

The microcapsules may contain insecticides, repellents, fungicides, anti-moss products, termite treatment products.

These microcapsules can be incorporated into numerous products, in particular phytosanitary products, paints, coatings, textiles, detergents, plastic materials, wood. The reaction mixture can be used directly, but it is also possible to use (especially in the case of microcapsules containing insecticides) washed, preferably isolated, optionally dried microcapsules. For all these uses, the microcapsules are advantageously of the fragile or porous type. They can be of the anionic type, but for repellents also of the cationic type. These microcapsules can find applications in the field of agriculture.

Other uses in the field of agriculture relate to microcapsules containing additives for the cultivation of plants, and/or fertilizers. Microcapsules of the fragile, porous or brittle type are used, which can be of the anionic or cationic type.

They are preferably washed.

Phytosanitary products can also be encapsulated by the process according to the invention; the reaction mixture can be used.

The microcapsules are advantageously of the fragile or porous type; they can be of the anionic or cationic type.

In the veterinary field, microcapsules can be used to encapsulate pharmaceutical or dermatological active principles, or else for the care of the coat, such as vermifuges, antiparasitics or agents for controlling or limiting odors. Advantageously, washed and dried microcapsules are used here, of the fragile, brittle, unbreakable or porous type, which may be anionic or cationic. They can in particular be incorporated into care products, pharmaceutical compositions, and food products; the incorporation of microcapsules containing pharmaceutical active ingredients into food products is a particularly interesting use.

The microcapsules can be used to treat or coat textile products or textile fibres, in particular technical ones. In this use, they may contain biocidal products, deodorizing products, self-cleaning products, self-healing products and/or self-repairing products. For all these uses, the reaction mixture can be used directly. The microcapsules can be of the fragile type, of the porous type (in particular for biocidal, deodorizing and self-cleaning products) or of the brittle type (in particular for self-healing and self-repairing products; with these two products one can also use microcapsules, washed or not). The microcapsules can be of the anionic or cationic type. These microcapsules can also be used to treat shoes, especially on the inside.

Still in the textile field, the microcapsules can contain detergent, softener, biocidal or antistatic products, anti-stain products; here, the reaction mixture can be used directly, and the microcapsules are advantageously of the porous or brittle type, and of the anionic and cationic type.

The microcapsules can be used in compositions for waterproof or water-repellent coatings; these coatings can be applied to textile surfaces or leather surfaces. The reaction mixture can be used directly. The microcapsules are preferably of the fragile or porous type and of the anionic type. They may contain hydrophobic compounds, in particular fatty compounds or fluorinated compounds.

The microcapsules can be used for the treatment of bedding (such as: mattresses, pillows, blankets, sheets, undersheets), luggage, fabrics and household equipment (such as: curtains, furniture with fabrics, furniture and other leather goods, cushions). For these uses, the microcapsules may contain biocidal products and/or repellent products (and in particular anti-mite products, anti-lice products, anti-mite products, anti-mosquito products), anti-stain products, perfumes, essential oils, fragrances and/or flavorings. For all these uses, the reaction mixture can be used directly. The microcapsules can be porous or brittle. They may be of the anionic type.

The microcapsules can be used in the formulation of deodorant and/or disinfectant products, and of anti-foam and antifungal products. The active substances are oily, natural or synthetic products. The microcapsules are preferably of the porous type and of the anionic type; the reaction mixture can be used directly.

Used in detergent products, the microcapsules can contain anti-foam products, enzymes and/or perfumes. The reaction mixture can be used directly. The microcapsules are preferably of the porous or brittle type, and of the anionic type.

The microcapsules can be used to coat building materials or thermal insulation products. For these uses, they may contain phase change materials (PCM) which provide effective thermal stabilization. These phase change materials may for example be active products based on paraffin or based on fatty acids (such as dodecanoic acid or esters, in particular methyl or decyl, of fatty acids), or else vegetable or animal waxes, such as beeswax. These active products advantageously have a melting point of between about 40° C. and about 100° C. For this use, the reaction mixture comprising the microcapsules can be used directly; thermal insulation products can also advantageously be prepared with dry microcapsules. For all these uses, the microcapsules can be of the unbreakable type. They may be of the anionic type; to coat construction materials they can also be of the cationic type.

Microcapsules containing phase change materials can also be used to cool batteries. For any use of phase change materials, the microcapsules must be of the unbreakable type, and their porosity must be as low as possible.

The microcapsules can be used in preparations ensuring watertightness, to be applied to solid materials (for example in the building sector) or flexible; in these preparations they may contain in particular sealing agents and/or expansion agents. The reaction mixture can be used directly. The microcapsules can be of the brittle type. They may be of the anionic type.

The microcapsules can contain lubricating and/or anti-friction products, or on the contrary non-slip products, essential oils, fragrances, perfumes and/or flavorings. These microcapsules can in particular be used on or in floor coverings. For all these uses, the reaction mixture can be used directly. The microcapsules can be of the brittle or unbreakable type. They may be of the anionic type.

The microcapsules may contain dyes and/or pigments. These microcapsules can be used in particular for dyeing with solvents, for transfer printing, for inkjet printing, for electrostatic printing, for screen printing or other printing techniques. For all these uses, the reaction mixture can be used directly. For solvent dyes, dried microcapsules can also be used. The microcapsules can be of the fragile or unbreakable type. They may be of the anionic type. In papermaking and printing, the reaction mixture can be used directly for coating paper, or for incorporation into a composition for making paper. For the manufacture of self-copying paper, microcapsules of the brittle type and of the anionic type are preferred. For scented impressions of the scratch & sniff or rub & sniff type, used for certain packaging materials, press articles or advertising products, it is also possible to use porous and unbreakable microcapsules; they are typically incorporated into inks.

It is possible to incorporate microcapsules according to the invention in inks and paints to manufacture road markings on the ground, panels or signage strips, in particular reflective. The reaction mixture can be used directly. The microcapsules are preferably of the unbreakable type, and of the anionic type.

The microcapsules can be used in self-healing compositions, which can be surface coatings used in paints, varnishes, inks, on rigid surfaces such as cement and concrete, and more generally on building materials, but also on flexible surfaces. Said self-healing compositions can also be incorporated into polymers and composites. The reaction mixture can be used directly, but in certain cases the residual impurities (in particular the residual amines) can interfere, and it is then necessary to wash the microcapsules.

The microcapsules are preferably of the porous type, and of the anionic type. If the self-repair must take place following an impact, microcapsules of the brittle type are referred.

A very particular use is that of self-repairing substances contained in washed and possibly dried microcapsules, used in the encapsulation materials of solar panels, and in particular for use in the aeronautical or aerospace field. These encapsulation materials are generally polymers. Typically, microcapsules of the brittle or unbreakable type are used, which may be, depending on the nature of the material in which they will be incorporated, of the anionic or cationic type.

The microcapsules can be used in compositions having a function of fire retardant and extinguisher. The reaction mixture can be used directly. The microcapsules are preferably of the brittle type and of the anionic type. They may contain brominated alkanes of general formula CₙH₂ₙ₊₂₋ₓBrₓThe microcapsules can contain thermochromic substances to detect and/or visualize the crossing of a temperature threshold, which has many applications such as in depilatory wax, packaging for food products to be heated, labels for refrigerated products making it possible to indicate a possible break in the cold chain, coatings allowing the indication of excessive stress or impacts following an accident, wear or misuse, in particular on plastic or composite parts, on mountaineering ropes, technical fabrics or protective helmets (motorcycle, bicycle, etc.). The reaction mixture can be used as it is, with unbreakable type microcapsules; they can be of the anionic or cationic type.

Unbreakable microcapsules having a thermochromic function can be used in holding and positioning guides and standards. They may be of the anionic type.

The microcapsules can contain essential oils, fragrances, perfumes, and/or flavorings, which can be used in many products, by mixing into the product or by applying a coating to the product. For all these uses, the reaction mixture can be used directly. The microcapsules can be of the brittle or unbreakable type. They may be of the anionic type.

More generally and following the same principles as above, are also concerned the fields of application such as antibacterial protection and protection against bad odors, uses in the field of the automobile and in particular of the passenger compartment, including the atmospheres of well-being and relaxation, maintenance of filters and decontaminants for military and civil sanitary applications, catalysts and surface modification agents.

For the decontamination of filters, in particular air filters, in particular for military decontamination, the reaction mixture can be used directly. The microcapsules are preferably porous type and anionic type.

In the automotive sector (automotive equipment, interior, accessories) the reaction mixture can be used directly. The microcapsules are preferably of the unbreakable type, and of the anionic type.

For technical catalysts and enzymes and surface modifiers, the reaction mixture can be used directly. The microcapsules are preferably of the brittle type and of the anionic type.

### Examples

To allow a person skilled in the art to reproduce the invention, examples of implementation are given here; they do not restrict the scope of the invention.

### Example 1: Preparation of Fragranced Microcapsules Based on a Diamine (HMDA)

(i) Preparation of the Emulsion

11.0 g of essential oil (Eucalyptus) was placed in a beaker, and the multi-acrylate monomer (Dipentaerythritol penta-/hexa-acrylate mixture) (0.39 g, 0.71 mmol) was dispersed in the essential oil under magnetic stirring (350 rpm). Stirring was maintained until the solution become homogeneous; a heating step was added if required. The essential oil/organic monomer assembly was added gradually to the previously prepared aqueous surfactant solution (40 g, PVA 2 wt.-%); the mixture was homogenized using an Ultraturrax™ IKA T10 at 9500 rpm for 3 min at ambient temperature to form an emulsion.

(ii) Microencapsulation

In a double-wall reactor, equipped with an IKA blade mechanical stirring system, preheated to 50° C., the previously prepared emulsion was introduced and stirred at a speed of 250 rpm. When the emulsion reached 50° C., the solution of diamine (Hexamethylene diamine HMDA) (0.17 g, 1.46 mmol) in 5 g of PVA 2 wt.-% solution was added dropwise using a syringe and under stirring (250 rpm). During the reaction, samples at different reaction times were taken and analyzed by optical microscopy and Fourier transform infrared (FTIR) spectroscopy in order to monitor the formation of the microcapsules.

The total quantity of monomers used was ˜0.56 g. The amine was used in excess with respect to the acrylate monomer so as to obtain a —NH/acrylate function ratio=1.6. The essential oil/water mass ratio is equal to 0.24.

The microcapsules can be analyzed by microscopy after a drying step. This analysis makes it possible to ensure the stability of the microcapsules once isolated. A second analysis consists of adding some drops of a fluorescent dye (Nile Red) on the dried microcapsules. Nile Red, a lipophilic chromophore which only fluoresces in an organic phase, makes it possible to verify that the core of the microcapsule still contains organic phase and that the microcapsules are filled.

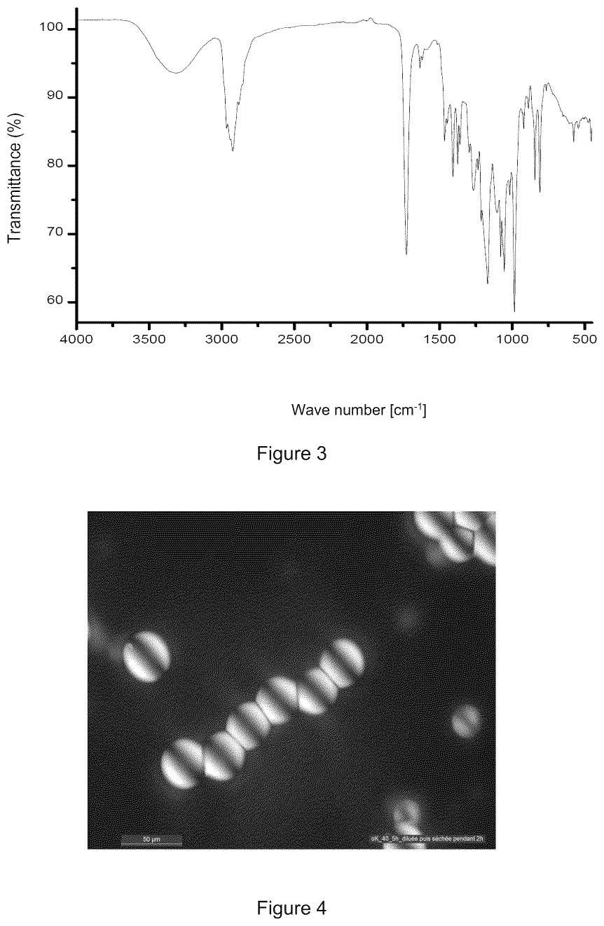

FIG. 2 shows an optical microscopy image of the reaction medium after 5 hours of reaction. The microcapsules are spherical, with a diameter between about 10 μm and about 25 μm. FIG. 3 shows the FTIR spectrum of the microcapsules isolated from a slurry after 6 hours of reaction (after washing with acetone, followed by three centrifugation cycles and oven drying). Characteristic vibrations of N—H bonds are observed around 3300 cm⁻¹ to 3400 cm⁻¹, along with a narrow band characteristic of a C═O bond around 1727 cm⁻¹.

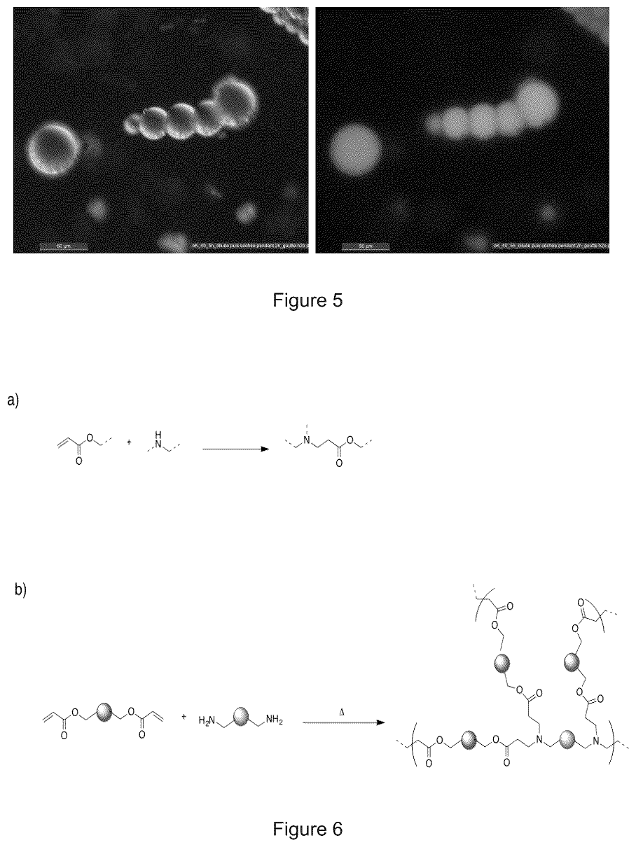

FIG. 4 shows an optical micrograph of microcapsules dried on a glass strip. The diameter thereof is around 30 μm to 35 μm. FIG. 5 shows a micrograph of microcapsules dried on a glass strip in glazing incidence light (on left) and in fluorescent light (on right) after adding some drops of Nile Red fluorescent dye. The intense emission in fluorescent light shows that the core of the microcapsule contains an organic phase.

### Example 2: Preparation of Fragranced Microcapsules Based on a Diamine (HMDA)

(i) Preparation of the Emulsion

11.0 g of a thermochromic solution (10° blue) was introduced into a beaker, placed in an oil bath and heated to 130° C. under magnetic stirring (350 rpm). Stirring was maintained until the thermochromic solution became homogeneous and transparent. The thermochromic solution was cooled, and when the temperature thereof reaches 50° C., the (multi)acrylate monomer (Dipentaerythritol penta-/hexa-acrylate mixture) (0.39 g, 0.71 mmol) is dispersed under magnetic stirring (350 rpm). Stirring is maintained until the solution becomes homogeneous. The thermochromic/organic monomer assembly was added gradually to the previously prepared aqueous surfactant solution (40 g, PVA 2 wt.-%); the mixture was homogenized using an Ultraturrax™ IKA T10 at 9500 rpm for 3 min at ambient temperature to form an emulsion.
(ii) Microencapsulation

In a double-wall reactor, equipped with an IKA blade mechanical stirring system, preheated to 50° C., the previously prepared emulsion was introduced and stirred at a speed of 250 rpm. When the emulsion reached 50° C., the solution of diamine (Hexamethylene diamine HMDA) (0.17 g, 1.46 mmol) in 5 g of PVA 2 wt.-% solution was added dropwise using a syringe and under stirring (250 rpm). During the reaction, samples at different reaction times were taken and analyzed by optical microscopy.

The total quantity of monomers used was˜0.56 g. The amine was used in excess with respect to the acrylate monomer so as to obtain a —NH/acrylate function ratio=1.6. The thermochromic solution/water mass ratio equals 0.24.

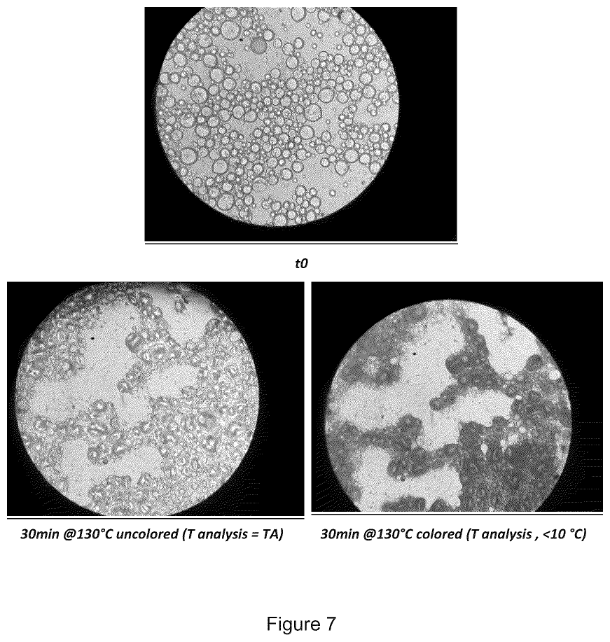

The dried microcapsules show a reversible color change with a reversible change of coloration at a temperature of 10° C. These same capsules can, furthermore, be heated in an oven at 130° C. for 30 min without modifying the thermochromic properties thereof (FIG. 7).

### Example 3: Poly(Beta-Amino Ester) Degradability Test

A first degradability test was performed according to the following procedure:
(1) Synthesis of poly(beta-amino ester)

In a beaker, the Hexamethylene diamine HMDA monomer (1.0 g, 8.6 mmol) was solubilized in THF (4.0 g) and added to a solution of (multi)acrylate (trimethylolpropane triacrylate) monomer (1.8 g, 6.1 mmol) solubilized in 2.5 g of THF. The mixture was placed in a pill box subsequently placed in an oil bath at 50° C.

The amine was used in excess with respect to the acrylate monomer so as to obtain a —NH/acrylate function ratio=2.

The polymer retrieved after 5 hours of reaction was washed three times with acetone and oven-dried.
(2) Poly(Beta-Amino Ester) Degradation

The degradation of the poly(beta-amino ester) was performed according to the following protocol:

20 mg of polymer solubilized in 1 mL of a sodium hydroxide solution (3M, in deuterated water D₂0, pH-14) is introduced into a flask equipped with a magnetic stirrer. As the polymer is crosslinked, it is not soluble in the aqueous phase.

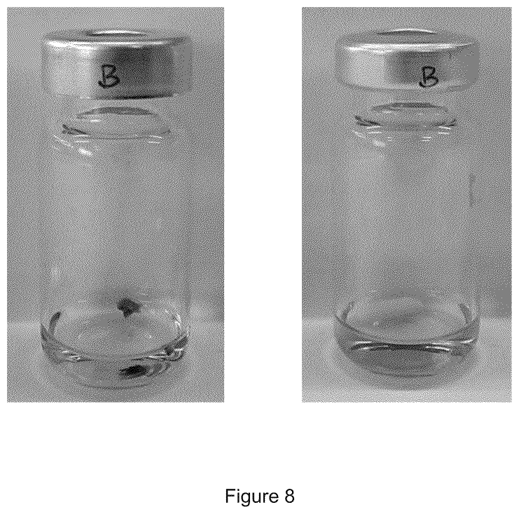

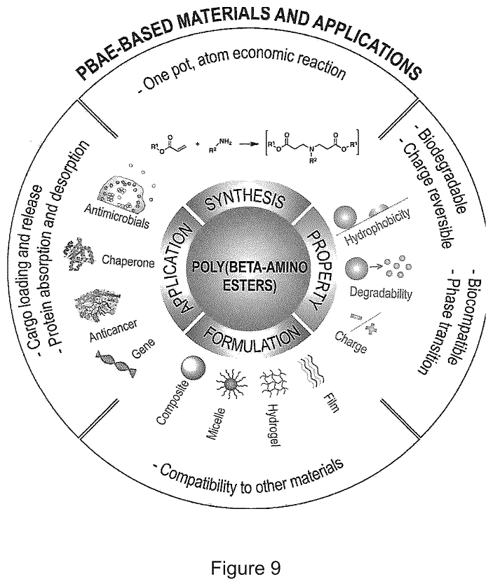

FIG. 8 shows that the poly(beta-amino ester) is dissolved in the aqueous phase, characterizing an effective degradation of the polymer under these accelerated degradation conditions.

### Example 4: Preparation of Fragranced Microcapsules Based on a Triamine (TREN)

(i) Preparation of the emulsion

11.0 g of essential oil (Eucalyptus) was placed in a beaker, and the multi-acrylate monomer (Dipentaerythritol penta-/hexa-acrylate mixture) (0.39 g, 0.74 mmol) was dispersed in the essential oil under stirring. The essential oil/organic monomer assembly was added gradually to the previously prepared aqueous surfactant solution (40 g, PVA 2 wt-.%); the mixture was homogenized using an Ultraturrax™ IKA T10 to form an emulsion.
(ii) Microencapsulation

In a double-wall reactor, equipped with an IKA blade mechanical stirring system, the previously prepared emulsion was introduced therein. An aqueous solution of tris(2-aminoethyl)amine TREN (0.145 g, 0.99 mmol) in 5 g of PVA 2 wt.-% solution was added under stirring at a temperature between 50° C. and 60° C.

### Example 5: Preparation of Thermochromic Microcapsules Based on a Triamine (TREN)

(i) Preparation of the emulsion

11.0 g of a thermochromic solution was introduced into a beaker and stirred hot, the multi-acrylate monomer (Dipentaerythritol penta-/hexa-acrylate mixture) (0.39 g, 0.74 mmol) was dispersed therein under stirring. The thermochromic /organic monomer assembly was added gradually to the previously prepared aqueous surfactant solution (40 g, PVA 2 wt.-%); the mixture was homogenized using an Ultraturrax™ IKA T10 to form an emulsion.
(ii) Microencapsulation

In a double-wall reactor, equipped with an IKA blade mechanical stirring system, the previously prepared emulsion was introduced at a temperature of about 50° C. to 60° C. An aqueous solution of tris(2-aminoethyl) amine TREN (0.145 g, 0.99 mmol) in 5 g of PVA 2 wt.-% solution was added under stirring at a temperature between 50° C. and 80° C.

### Example 6: Preparation of Microcapsules Based on a Biogenic Monomer

(i) Preparation of the Emulsion

11.0 g of essential oil (Eucalyptus) was placed in a beaker, and the multi-acrylate monomer (Dipentaerythritol penta-/hexa-acrylate mixture) (0.39 g, 0.74 mmol) was dispersed in the essential oil under stirring. The essential oil/organic monomer assembly was added gradually to the previously prepared aqueous surfactant solution (40 g, PVA 2 wt.-%); the mixture was homogenized using an Ultraturrax™ IKA T10 to form an emulsion.
(ii) Microencapsulation

In a double-wall reactor, equipped with an IKA blade mechanical stirring system, the previously prepared emulsion was introduced, the aqueous solution of diamine (Butane-1,4-diamine (Putrescine)) (0.13 g, 1.47 mmol) in 5 g of PVA 2 wt % was added under stirring at a temperature between 50° C. and 60° C.

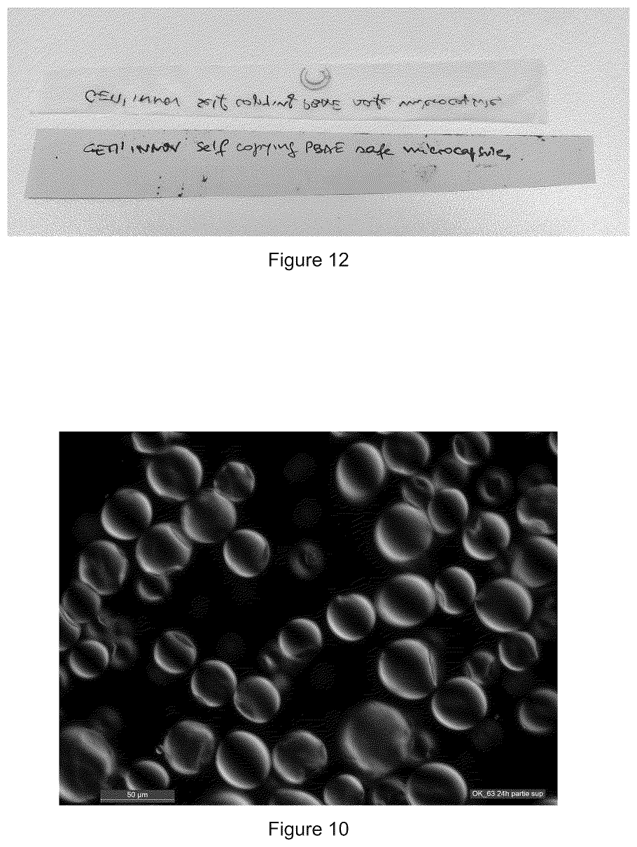

FIG. 10 shows an optical microscopy image of the capsules after 24 hours of reaction. The microcapsules are spherical, with a mean diameter between about 10 μm and about 30 μm.

### Example 7: Preparation of Microcapsules Based on Polyethylene Imine (PEI)

(i) Preparation of the Emulsion

11.0 g of essential oil (Eucalyptus) was placed in a beaker, and the multi-acrylate monomer (Dipentaerythritol penta-/hexa-acrylate mixture) (0.39 g, 0.74 mmol) was dispersed in the essential oil under stirring. The essential oil/organic monomer assembly was added gradually to the previously prepared aqueous surfactant solution (40 g, PVA 2 wt.-%); the mixture was homogenized using an Ultraturrax^{M} IKA T10 to form an emulsion.
(ii) Microencapsulation

In a double-wall reactor, equipped with an IKA blade mechanical stirring system, the previously prepared emulsion was introduced. A solution of polyethylene imine (PEI) (1.78 g, 1.48 mmol) in 5 g of PVA 2 wt.-% solution was added under stirring at a temperature between 50° C. and 60° C.

FIG. 11 shows optical microscopy images of the capsules after 24 hours of reaction. The microcapsules are spherical, with a mean diameter between about 10 μm and about 30 μm.

### Example 8: Preparation of Fragranced Microcapsules (Shell/PI Ratio=3.4%)

(i) Preparation of the Emulsion

193.6 g of essential oil (Eucalyptus) was placed in a beaker, and the multi-acrylate monomer (Dipentaerythritol penta-/hexa-acrylate mixture) (4.5 g, 8.5 mmol) was dispersed in the essential oil under stirring. The essential oil/organic monomer assembly was added gradually to the previously prepared aqueous surfactant solution (255.9 g PVA 2 wt.-%); the mixture was homogenized to form an emulsion.
(ii) Microencapsulation

In a double-wall reactor, equipped with an IKA blade mechanical stirring system, the previously prepared emulsion was introduced. A solution of diamine (Hexamethylene diamine HMDA) (2.01 g, 17.2 mmol) in 44.1 g of a PVA 2 wt.-% solution was added under stirring at a temperature between 50° C. and 60° C. The whole was left to react for 2 hours at 50° C. and for 5 hours at 60° C.

### Example 9: Preparation of Fragranced Microcapsules

(i) Preparation of the Emulsion

11.0 g of a mixture of 80% Pineapple papaya fragrance (reference RS42370 from the company Technicoflor in Allauch (France)) and 20% methyl myristate was placed in a beaker, and the multi-acrylate monomer (Dipentaerythritol penta-/hexa-acrylate mixture) (0.39 g, 0.74 mmol) was dispersed in the fragrance under stirring. The essential oil/organic monomer assembly was added gradually to the previously prepared aqueous surfactant solution (40 g, PVA 2 wt.-%); the mixture was homogenized using an Ultraturrax™ IKA T10 to form an emulsion.
(ii) Microencapsulation

In a double-wall reactor, equipped with an IKA blade mechanical stirring system, the previously prepared emulsion was introduced. A solution of diamine (Hexamethylene diamine HMDA) (0.17 g, 1.49 mmol) in 5 g of a PVA 2 wt.-% solution was added under stirring at a temperature between 50° C. and 60° C. The whole was left to react for 2 hours at 50° C. and for 5 hours at 60° C.

### Example 10: Preparation of Microcapsules for Carbonless Copy Papers (Shell/PI Ration=3.4%)

(i) Preparation of the Emulsion

193.6 g of an internal phase (Dye) was placed in a beaker, and the multi-acrylate monomer (Dipentaerythritol penta-/hexa-acrylate mixture) (4.5 g, 8.5 mmol) was dispersed in the internal phase under stirring. The whole was added gradually to the previously prepared aqueous surfactant solution (255.9 g PVA 2 wt.-%); the mixture was homogenized to form an emulsion.
(ii) Microencapsulation

In a double-wall reactor, equipped with an IKA blade mechanical stirring system, the previously prepared emulsion was introduced. An aqueous solution of diamine (Hexamethylene diamine HMDA) was added, under stirring at a temperature between 50° C. and 60° C.
(iii) Use of the Microcapsules in a Carbonless Copy Paper

These microcapsules were applied on a sheet of paper, according to known methods, and used in a carbonless copy system. FIG. 12 shows the result, which is fully satisfactory.

### Example 11: Preparation of Thermochromic Microcapsules Based on POSS@Octa(Acrylate) Monomer

(i) Preparation of the Emulsion

20.0 g of thermochromic, and polyoctahedral silsesquioxanes borne by eight acrylate functions (POSS@octa(acrylate), CAS No. 1620202-27-8, purchased from Hydridplastics, 1.48 g, 1.12 mmol) and Butylated HydroxyToluene (BHT, 5.0 mg) thermal inhibitor, were placed in a beaker. The mixture was solubilized hot under magnetic stirring. Stirring was maintained until the solution became homogeneous. The thermochromic/POSS@octa(acrylate) assembly was added gradually to the previously prepared aqueous surfactant solution (40 g, PVA 2 wt.-%); the mixture was homogenized using an Ultraturrax™ IKA T10 to form an emulsion.
(ii) Microencapsulation

In a reactor, the previously prepared emulsion was introduced.

The solution of Hexamethylene diamine (HMDA, 0.35 g, 3.01 mmol) in water was added dropwise using a syringe and under stirring. The whole was left to react at 50° C. for 1 hour and at 80° C. for 23 hours.

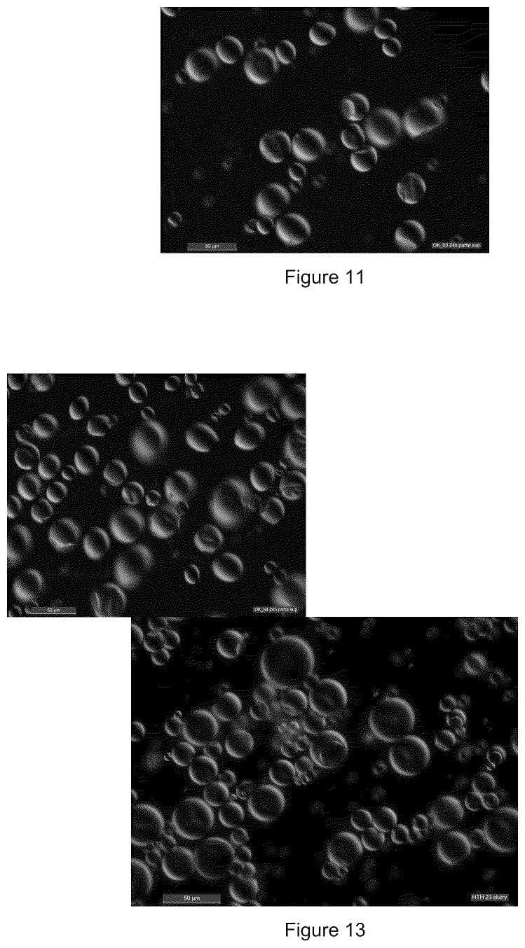

FIG. 13 shows a photograph of these microcapsules.

### Example 12: Preparation of thermochromic microcapsules based

on POSS@octa(acrylate) monomer with meta-xylylenediamine (i) Preparation of the emulsion 10.0 g of thermochromic, and polyoctahedral silsesquioxanes borne by eight acrylate functions (POSS@octa(acrylate), CAS

No. 1620202-27-8, purchased from Hydridplastics, 1.50 g, 1.12 mmol) and Butylated HydroxyToluene (BHT, 5.0 mg) thermal inhibitor, were placed in a beaker. The mixture was solubilized hot under magnetic stirring. Stirring was maintained until the solution became homogeneous. The thermochromic/POSS@octa(acrylate) assembly was added gradually to the previously prepared aqueous surfactant solution (40 g, PVA 2 wt.-%); the mixture was homogenized using an Ultraturrax™ IKA T10 to form an emulsion.
(ii) Microencapsulation

In a reactor, the previously prepared emulsion was introduced. The solution of meta-xylylenediamine (CAS No. 1477-55-0, 0.60 g, 3.01 mmol) in 3 mL of water was added dropwise using a syringe and under stirring. The whole was left to react at 65° C. for 1 hour and at 80° C. for 17 hours.

### Example 13: Preparation of Thermochromic Microcapsules Based on POSS@Octa(Acrylate) Monomer with POSS@Octammonium and Hexamethylene Diamine (HDMA)

(i) Preparation of the Emulsion

10.0 g of thermochromic, and polyoctahedral silsesquioxanes borne by eight acrylate functions (POSS@octa(acrylate), CAS No. 1620202-27-8, purchased from Hydridplastics, 1.40 g, 1.06 mmol) and Butylated HydroxyToluene (BHT, 5.0 mg) thermal inhibitor, were placed in a beaker. The mixture was solubilized hot under magnetic stirring. Stirring was maintained until the solution became homogeneous. The thermochromic/POSS@octa(acrylate) assembly was added gradually to the previously prepared aqueous surfactant solution (40 g, PVA 2 wt.-%); the mixture was homogenized using an Ultraturrax™ IKA T10 to form an emulsion. (ii) Microencapsulation

In a reactor, the previously prepared emulsion was introduced. Afterward, the solution of Hexamethylene diamine (HMDA, 0.70 g, 6.02 mmol), POSS@(octa)ammonium (CAS No. 150380-11-3, purchased from Hydridplastics, 0.30 g, 0.26 mmol), and potassium carbonate (0.16 g, 1.16 mmol) in water was added dropwise using a syringe, under stirring. The whole was left to react at 65° C. for 1 hour and at 80° C. for 17 hours.

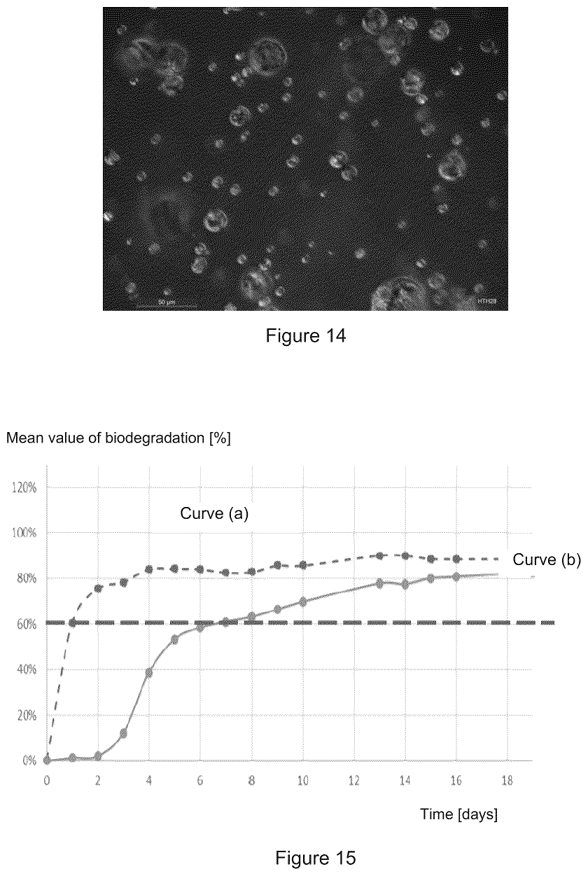

FIG. 14 shows a photograph of these microcapsules.

### Example 14: Biodegradation Test

A batch of microcapsules prepared according to example 8 was provided. The dry microcapsules but containing essential oil (Eucalyptus) were subjected to the biodegradability test described in the document OECD 301 (“OECD Guidelines for Testing of Chemicals: Ready Biodegradability”) using method 301 F (Manometric respirometry test). After an incubation time of nineteen days, the biodegradation percentage was 83%.

FIG. 15 shows the progression of the biodegradation percentage as a function of time, over a 19-day duration. Curve (b) corresponds to the microcapsule, while curve (a) corresponds to a reference product (sodium acetate) processed separately under the same biodegradation conditions.

### Example 15: Biodegradation Test

A batch of microcapsules prepared according to example 8 was provided. The microcapsules were opened, emptied and washed. Then they were subjected to the biodegradability test described in the document OECD 301 (“OECD Guidelines for Testing of Chemicals: Ready Biodegradability”) using method 301 F (Manometric respirometry test). After an incubation time of twenty-eight days, the biodegradation percentage was 93%.

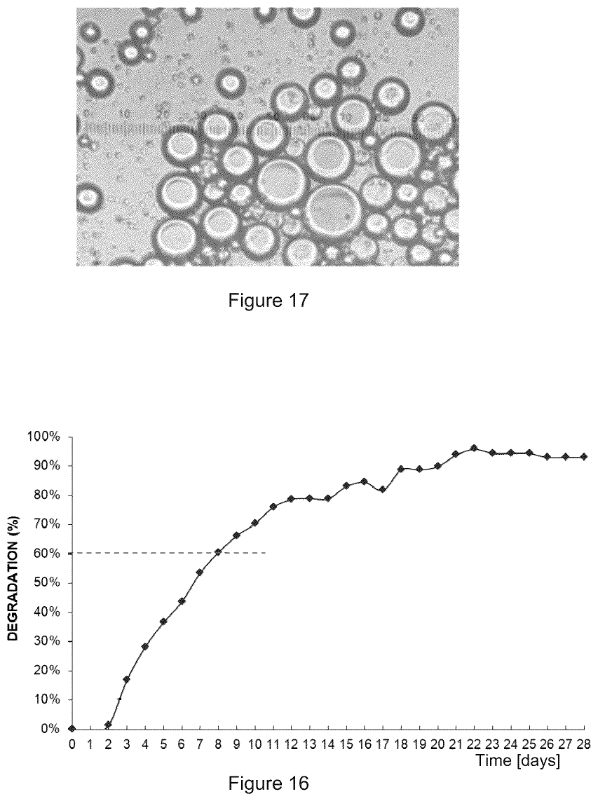

FIG. 16 shows the progression of the biodegradation percentage as a function of time.

### Example 16: Preparation of Fragranced Microcapsules Based on a Multiamine (Pentaethylenehexamine)

(i) Preparation of the emulsion

19.7 g of essential oil (Eucalyptus) was placed in a beaker, and the multi-acrylate monomer (Dipentaerythritol penta-/hexa-acrylate mixture) (1.2 g, 2.29 mmol) was dispersed in the essential oil under magnetic stirring (350 rpm) at 50° C. Stirring was maintained until the solution became homogeneous. The essential oil/organic monomer assembly was added gradually to the prepared aqueous surfactant solution (31.7 g, PVA 2 wt.-%) previously heated to 50° C.; the mixture was homogenized using an Ultraturrax™ IKA T10 at 11,500 rpm for 3 min at 50° C. to form an emulsion.
(ii) Microencapsulation

In a double-wall reactor, equipped with an IKA blade mechanical stirring system, preheated to 50° C., the previously prepared emulsion was introduced and stirred at a speed of 250 rpm. The solution of multiamine (Pentaethylenehexamine) (1.9 g, 8.00 mmol) in 5.5 g of PVA 2 wt.-% solution was added dropwise using a syringe and under stirring (250 rpm). The reaction mixture was kept under stirring for 2 hours at 50° C. then 5 hours at 60° C. The total quantity of monomers used was 3.1 g. The amine was used in excess with respect to the acrylate monomer so as to obtain an Amine/acrylate molar ratio=3.5. The essential oil/water mass ratio is equal to 0.53.

### Example 17: Preparation of Fragranced Microcapsules Based on an Aromatic Diamine (m-Xylylene Diamine)

(i) Preparation of the Emulsion

22.0 g of fragrance was placed in a beaker, and the multi-acrylate monomer (Dipentaerythritol penta-/hexa-acrylate mixture) (1.52 g, 2.90 mmol) was dispersed in the fragrance under magnetic stirring (350 rpm) at 50° C. Stirring was maintained until the solution became homogeneous. The fragrance/organic monomer assembly was added gradually to the previously prepared aqueous surfactant solution (35.0 g, PVA 2 wt.-%); the mixture was homogenized using an Ultraturrax™ IKA T10 at 11,500 rpm for 3 min at 50° C. to form an emulsion.
(ii) Microencapsulation

In a double-wall reactor, equipped with an IKA blade mechanical stirring system, preheated to 65° C., the previously prepared emulsion was introduced and stirred at a speed of 250 rpm. When the emulsion has reached 65° C., the solution of m-xylylenediamine (0.80 g, 5.88 mmol) in 5.0 g of PVA 2 wt.-% solution was added dropwise using a syringe and under stirring (248 rpm). The reaction mixture is kept under stirring for 5 hours at 65° C. and 1 hour at 80° C.

The total quantity of monomers used was 2.3 g. The amine was used in excess with respect to the acrylate monomer so as to obtain a —NH/acrylate function ratio=1.6. The fragrance/water mass ratio is equal to 0.55.

FIG. 17 shows a photograph of these microcapsules.

### Example 18: Preparation of Fragranced Microcapsules with a Cellulose Fiber Coating

(i) Preparation of the Emulsion

22.0 g of fragrance was placed in a beaker, and the multi-acrylate monomer (Dipentaerythritol penta-/hexa-acrylate mixture) (1.52 g, 2.90 mmol) was dispersed in the fragrance under magnetic stirring (350 rpm) at 50° C. Stirring was maintained until the solution became homogeneous. The fragrance/organic monomer assembly was added gradually to the previously prepared aqueous surfactant solution (40.0 g, PVA 2 wt.-%); the mixture was homogenized using an Ultraturrax™ IKA T10 at 11,500 rpm for 3 min at 50° C. to form an emulsion.
(ii) Microencapsulation

In a double-wall reactor, equipped with an IKA blade mechanical stirring system, preheated to 65° C., the previously prepared emulsion was introduced and stirred at a speed of 250 rpm. When the emulsion reached 65° C., the solution of m-xylylenediamine (0.80 g, 5.88 mmol) in 5.0 g of PVA 2 wt.-% solution was added dropwise using a syringe and under stirring (250 rpm). The reaction mixture is kept under stirring for 5 hours at 65° C. and 1 hour at 80° C.

The total quantity of monomers used was 2.3 g. The amine was used in excess with respect to the acrylate monomer so as to obtain a —NH/acrylate function ratio=1.6. The essential oil/water mass ratio is equal to 0.5.
(iii) Cellulose Coating

4 wt.-% of cellulose microfiber (Exilva F 01-L) was preheated to a temperature between 65° C. and 70° C. then introduced into the hot slurry under stirring. The mixture is homogenized hot under stirring for 30 min and for 2 hours at ambient temperature.

A cotton fiber bonding test was performed: a cotton fiber was previously wetted and then steeped in the slurry. After washing vigorously and thoroughly in water to simulate rinsing, the fiber was dried at ambient temperature.

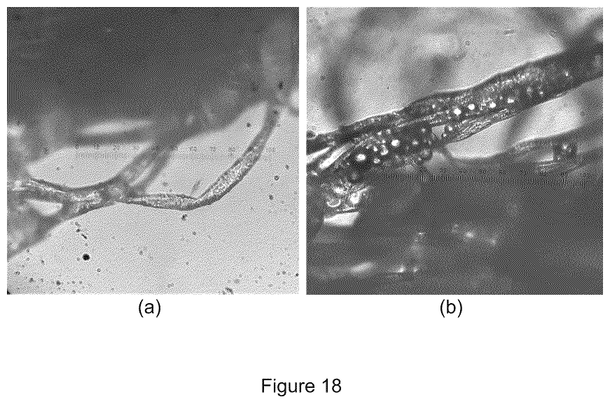

FIG. 18 shows a photograph (image (b)) of a cotton fiber after steeping in a slurry solution then drying for microcapsules in which the surface has been modified. The coating enhances the bonding of the microcapsules on the cotton fiber, compared to uncoated microcapsules (image (a)).

### Example 19: Preparation of Microcapsules (Brittle Type) Based on Hexamethylene-Diamine and Dipentaerythritol Penta/Hexa-Acrylate

(i) Preparation of the Emulsion

In a 250 ml beaker, dipentaerythritol penta-/hexa-acrylate (2.9 g) (CAS No. 60506-81-2) was dispersed under magnetic stirring (350 rpm) at 50° C. in a perfume (80.0g). Stirring was maintained until the solution became homogeneous (solution “A”). Solution “A” was added gradually to the previously prepared aqueous surfactant soluton (105.5 g of PVA (8-88) at 2.0% by weight) in a beaker. The mixture was homogenized using a HOMOMIXER at 5000 rpm for 3 min at 50° C. to form an emulsion.
(ii) Microencapsulation

This beaker containing the emulsion was placed in a water bath heated to 50° C., and the emulsion was placed under mechanical stirring with IKA blades (250 rpm). The solution of hexametylenediamine (CAS No: 124-09-4) (1.3 g) in a 2.0 wt.-% PVA solution (18.2 g) was added dropwise using a separatory funnel. Solution “B” composed of microcapsules at the start of polymerization is obtained. Solution B was left stirring at 50° C. for 18 hours; cooling was done under stirring at room temperature.

These microcapsules are of the fragile type.

Example 20:

POSS: Preparation of microcapsules (unbreakable type) based on meta-xylylenediamine and Polyhedral Oligomeric Silsesquioxane (POSS®)
(i) Preparation of the emulsion

In a beaker, the Polyhedral Oligomeric Silsesquioxane (6.0 g) was dispersed under magnetic stirring (350 rpm) at 50° C. in the perfume (36.1 g). Stirring was maintained until the solution became homogeneous (solution “A”). Solution A was gradually added to a beaker containing the previously prepared aqueous surfactant solution (144.3 g, PVA (8-88) at 2.0 wt.-%). The mixture was homogenized using a HOMOMIXER at a speed of 5000 rpm for 3 min and at a temperature of 50° C. to form an emulsion.
(ii) Microencapsulation

This beaker containing the emulsion was placed in a water bath heated to 50° C., and the emulsion was placed under mechanical stirring with IKA blades (250 rpm). The solution of meta-xylylenediamine (CAS No: 1477-55-0) (2.5 g) in deionized water (10.8 g) was added dropwise using a separation funnel. Solution “B” composed of microcapsules at the start of polymerization is obtained. Solution B was left stirring at a temperature of 50° C. for 1 hour then 65° C. for 5 hours. Cooling was done under stirring at room temperature.

These microcapsules are of the unbreakable type.

### Example 21: Preparation of PBAE Microcapsules with Temperature Cycling

(i) Preparation of the emulsion

1,140.5 g of perfume was placed in a beaker, and the multi acrylate monomer (Dipentaerythritol penta-/hexa-acrylate mixture) (67.9 g) was dispersed in the perfume under stirring at 50° C. Stirring was continued until the solution became homogeneous. This solution was added gradually to a previously prepared aqueous surfactant solution (2,073.7 g, PVA 2 wt.-%); the mixture was homogenized using a Homomixer at 4,000 rpm for 3 min at 50° C. to form an emulsion.
(ii) Microencapsulation

In a double-wall reactor, equipped with an IKA mechanical blade stirring system, preheated to 50° C., the previously prepared emulsion was introduced and stirred at a speed of 550 rpm. The solution of meta-xylylenediamine (31.11 g) in 259.21 g of PVA solution (2% by weight) was added dropwise under stirring. The reaction mixture is kept under stirring for 1 hour at 50° C., 4 hours at 65° C. and 1 hour at 80° C.

### Example 22: Preparation of Scented Microcapsules Coated with the Amino Acid Derivative N-Acetylglycine and Cellulose Fiber (“Soft” Version)

The reaction mixture (slurry) obtained according to Example 21 was left to stand at room temperature for 24 h. A quantity of 1,000 g of this slurry was introduced into a reactor equipped with an IKA mechanical blade stirring system. Then the slurry was heated to 75° C. and stirred at a speed of 550 rpm. When the slurry had reached 75° C., a solution at 95° C. of a mixture of N-acetylglycine (10 g) in a previously prepared 2 wt.-% PVA solution (50 g) was added thereto under stirring. The reaction mixture was kept under stirring for 5 min at 75° C., then allowed to cool to 50° C. under stirring. Then, 187 g of cellulose microfiber (Exilva F 01-L) was preheated to 50° C. and then introduced into the slurry under stirring. The mixture was homogenized at 50° C. under stirring for 30 min at 50° C., then for 2 h at room temperature.

### Example 23: Preparation of Scented Microcapsules Coated with the Amino Acid Derivative N-Acetylglycine and Cellulose Fiber (“Medium” Version)

The slurry obtained according to Example 21 was left to stand at room temperature for 24 hours.

A quantity of 206 g of this slurry was introduced into a reactor equipped with an IKA mechanical blade stirring system. Then the slurry was heated to 75° C. and stirred at a speed of 550 rpm. When the slurry had reached 75° C., a solution at 95° C. of a mixture of N-acetylglycine (2.6 g) in a previously prepared 2 wt.-% PVA solution (11.8 g) was added under stirring (550 rpm). The reaction mixture was kept under stirring for 5 min at 75° C., then allowed to cool to 50° C. under stirring. Then, 38.8 g of cellulose microfiber (Exilva F 01-L) was preheated to 50° C. and then introduced into the slurry under stirring. The mixture was homogenized at 50° C. under stirring for 30 min at 50° C., then for 2 h at room temperature.

### Example 24: Preparation of Scented Microcapsules with a Coating with the Derivative of the Amino Acid N-Acetylglycine and the Cellulose Fiber (“Hard” Version)

The slurry obtained according to Example 21 was left to stand at room temperature for 24 hours. A quantity of 1,000 g of this slurry was introduced into a reactor equipped with an IKA mechanical blade stirring system. Then the slurry was heated to 75° C. and stirred at a speed of 550 rpm. When the slurry had reached 75° C., a solution at 95° C. of N-acetylglycine (15 g) in a previously prepared PVA solution (75 g, 2 wt.-%) was added under stirring (550 rpm). The reaction mixture was kept under stirring for 5 min at 75° C., then allowed to cool to 50° C. under stirring. Then, 192.3 g of cellulose microfiber (Exilva F 01-L) was preheated to 50° C. and then introduced into the slurry under stirring. The mixture was homogenized at 50° C. under stirring for 30 min at 50° C., then for 2 h at room temperature.

Table 1 below summarizes the mechanical properties of microcapsules obtained according to Examples 22, 23 and 24.

TABLE 1
Mechanical properties of rupture of scented microcapsules coated
with amino acid derivative N-acetylglycine and cellulose fiber

| | SOFT | Medium | Hard |
|---|---|---|---|
| | (Example 22) | (Example 23) | (Example 24) |
| Size [μm] | 20.3 ± 0.7 | 22.2 ± 0.5 | 25.5 ± 0.6 |
| Force at break | 0.91 ± 0.07 | 1.20 ± 0.08 | 1.90 ± 0.16 |
| [mN] | | | |
| Deformation at | 43.5 ± 1.1 | 40.4 ± 1.4 | 48.4 ± 1.4 |
| break [%] | | | |
| Number of | 50 | 50 | 50 |
| microcapsules | | | |
| measured | | | |

Example 25: Preparation of scented microcapsules coated with N-acetylglycine, alginate and cellulose fiber

The microcapsule slurry was prepared as described in Example 21, replacing meta-xylylenediamine with hexamethylene diamine. It was left to stand at room temperature for 24 h. A quantity of 40 g of this slurry was introduced into a reactor equipped with an IKA mechanical blade stirring system. Then the slurry was heated to 75° C. and stirred at a speed of 550 rpm. When the slurry reached 75° C., a previously prepared solution at 95° C. of a mixture of N-acetylglycine (0.4 g) in water (2.5 g) was added under stirring (550 rpm). The reaction mixture was kept under stirring for 2 min at 75° C. Then, sodium alginate (0.12 g) was added, and the reaction mixture was cooled to 40° C. under stirring. Then, the cellulose microfiber (4 g, Exilva F 01-L) was introduced into the reaction reactor and kept stirring for 30 min. Finally, a solution of calcium chloride (0.4 g) in water was added to the slurry. The mixture was homogenized at 40° C. under stirring for 30 min at 40° C. and for 2 h at room temperature.

Examples 26 to 29 which follow relate to microcapsules whose outer shell has been modified by various means: radical polymerization (example 26), thermal polymerization in the presence of an initiator (example 27), photopolymerization (example 28) or quaternization (example 29).

### Example 26: Redox Polymerization by Potassium Persulfate in the Presence of Iron (II) Sulfate

20 g of a flavored slurry were placed in a 100 ml three-necked flask under magnetic stirring at 35° C. This slurry was then degassed with argon for 30 min. In a hemolysis tube, an aqueous solution of potassium persulfate (KPS) was prepared and then degassed for 5 min (30 mg of KPS in 1 ml of water). 9 mg of iron (II) sulfate FeSO₄ were dissolved in 1 ml of H₂O in another hemolysis tube, and the solution is then degassed with argon for 5 min.

The KPS solution was removed and added to the slurry dispersion under magnetic stirring at 250 rpm at 35° C., and likewise, the FeSO₄ solution was added thereto. The reaction mixture was homogenized at 35° C. for 3 h.

The capsules were dried and then analyzed by thermogravimetric analysis (TGA), see FIG. 21. In order to characterize a given sample, the onset temperature (Tₒnsₑₜ) which indicates the temperature at which mass loss begins was calculated.

The following results were found: Tₒₙₛₑₜ is 177.5° C. for unmodified microcapsules, and 196.6° C. for KPS-modified microcapsules.

### Example 27: Thermal Polymerization in the Presence of the Lipophilic Initiator AIBN

(i) Preparation of the emulsion

In a 60 ml flask, a solution of essential oil (22 g), the multi acrylate monomer (Dipentaerythritol penta-/hexa-acrylate mixture) (1.52 g) and the liposoluble initiator azobisisobutyronitrile (AIBN, 0.03 g) were mixed and then degassed with argon for 15 min under magnetic stirring at 30° C.

An aqueous solution of PVA (35 g, 2 wt.-%) was prepared and degassed with argon in a 60 ml flask for 15 min, the two solutions previously prepared and degassed were heated up to 50° C.

The PVA solution was quickly transferred to a beaker then the essential oil solution was added thereto, and the mixture was homogenized using an Ultra-turrax IKA T10 for 3 min at 50° C. to form an emulsion. This emulsion was transferred to a double-wall reactor equipped with an IKA mechanical blade stirring system and preheated to 50° C.; the emulsion was degassed with argon.
(ii) Microencapsulation

The solution of Hexamethylene diamine (HMDA, 0.5 g) in 5 g of 2 wt % PVA solution was added dropwise using a syringe and under mechanical stirring (250 rpm). The reaction takes place for 1 hour at 50° C. and 4 hours at 65° C. then 1 hour at 80° C. The mixture was homogenized at 50° C. for 1 hour, then at 65° C. for 4 hours and at 80° C. for 1 hour.

The capsules were dried and then analyzed by thermogravimetric analysis (TGA), see FIG. 22. In order to characterize a given sample, the onset temperature (Tₒₙₛₑₜ) which indicates the temperature at which mass loss begins was calculated.

The following results were found:

Tₒₙₛₑₜ is 177.5° C. for unmodified microcapsules, and 187.7° C. for AIBN-modified microcapsules.

### Example 28: Photopolymerization

(i) Preparation of the emulsion

In a beaker, 0.015 g of the liposoluble photoinitiator Diphenyl (2,4,6-trimethylbenzoyl) phosphine oxide (TPO) were dissolved in 22 g of essential oil at 30° C. for 10 min, the multi acrylate monomer (mixture Dipentaerythritol penta/hexa-acrylate) (1.52 g) was then added with stirring. The mixture obtained was added gradually to the previously prepared aqueous surfactant solution (35 g, PVA 2 wt.-%); the mixture was homogenized using an Ultra turrax™ IKA T10 at 9500 rpm for 3 min at 50° C. to obtain an emulsion.
(ii) Microencapsulation

In a double-wall reactor, equipped with a mechanical stirring system with IKA blades, preheated to 50° C., the previously prepared emulsion was introduced and stirred at a speed of 250 rpm.

The solution of Hexamethylene diamine (HMDA, 0.5 g) in 5 g of PVA solution (2 wt.-%) was added dropwise using a syringe and with mechanical stirring (250 rpm) for 1 hour at 50° C., 4 hours at 65° and 1 hour at 80° C.
(iii) Photopolymerization

3 ml of microcapsules obtained were placed in a tank then irradiated at 365 nm using a UV lamp for 5 min, at the end of the photopolymerization, the capsules were dried and then analyzed by ATG, see FIG. 23.

Tₒₙₛₑₜ is 160.1° C. for unmodified microcapsules, and 189.1° C. for irradiation-modified microcapsules.

### Example 29: Quaternization

50 g of a previously prepared slurry of perfumed microcapsules were placed in a three-necked flask (250 ml). The pH of the slurry was then lowered to 3-4 by adding acrylic acid. The mixture was then degassed with argon for 30 min, under magnetic stirring (300 rpm) at 35° C. An aqueous solution of potassium persulfate (KPS) was prepared; it was degassed for 5 min (65.5 mg of KPS in 2.5 ml of water). 19.4 mg of iron (II) sulfate FeSO₄ was dissolved in 2.5 ml of water in another glass vial, and this solution was then degassed with argon for 5 min.

The KPS solution was added to the mixture (slurry+acrylic acid) under magnetic stirring at 300 rpm at 350 C, then the FeSO₄ solution was added. The reaction mixture was homogenized at 35b C for 3 h.

Table 2 gives the classification of the microcapsules resulting from examples 1 to 29.

TABLE 2
Summary of properties of microcapsules

| Example | Fragile/soft | Brittle | Unbreakable | Remark |
|---|---|---|---|---|
| 1 | X | | | HMDA with penta/hexa acrylate |
| 2 | X | | | HMDA with penta/hexa acrylate |
| 4 | | X | | TREN with penta/hexa acrylate |
| 5 | | X | | TREN with penta/hexa acrylate |
| 6 | X | | | Putrescine (diamine) with |
| | | | | penta/hexa acrylate |
| 7 | | X | | PEI with penta/hexa acrylate |
| 8 | X | | | HMDA with penta/hexa acrylate |
| 9 | X | | | HMDA with penta/hexa acrylate |
| 10 | | X | | Use: self-copying paper |
| 11 | | | X | POSS/thermochromic |
| 12 | | | X | POSS |
| 13 | | | X | POSS |
| 16 | | X | | Multiamine |
| 17 | X | | | m-xylene diamine |
| 18 | | | X | With cellulose coating |
| 19 | X | | | HMDA with penta/hexa acrylate |
| 20 | | | X | (POSS marque incassable) |
| 21 | X | | | m-xylene diamine |
| 22 | X | | | Version « soft » ACG |
| 23 | | X | | Medium Version « medium » ACG |
| 24 | | X | | Version « hard » ACG |
| 25 | | | X | Multicoating cellulose/alginate |
| 26 | | X | | Radical KPS |
| 27 | | X | | AIBN |
| 28 | | X | | Photoirradiation |
| 29 | | X | | quaternization |

Table 3 gives examples of applications that have been tried.

TABLE 3
Examples of use for different types of microcapsules

| Use | Fragile | Brittle | Unbreakable | Preferred technical functions |
|---|---|---|---|---|
| Detergents, | x | x | | The capsule must release its |
| Fabric | | | | content (perfume) when a certain |
| softeners. | | | | force is exerted after washing and |
| | | | | when the user handles the textile. |
| | | | | The stability in its container must |
| | | | | exceed a minimum time fixed by the |
| | | | | specifications, without being |
| | | | | altered. |
| Fragrance | X | X | X | The capsule must release its |
| and | | | | content according to either a |
| perfums. | | | | programmed use (liquid release time |
| Essential | | | | provided for in a specification) or |
| oils. | | | | by diffusion without breaking the |
| Aromas. | | | | capsule, or even instantly when the |
| | | | | capsule is broken under the action |
| | | | | of an external force. |
| Biocides. | X | | X | The capsule must release the active |
| Insecticides. | | | | agent in liquid form over a given |
| Repellants. | | | | time (X months) or must deliver the |
| Termite | | | | active agent without breaking the |
| treatment. | | | | capsule shell. The release and/or |
| | | | | delivery time of the active agent |
| | | | | depends on the specifications; it |
| | | | | must, in addition to a duration, |
| | | | | take into account the ambient |
| | | | | environment (pH, humidity, |
| | | | | temperature, altitude + other |
| | | | | factors: seasons, latitude and |
| | | | | longitude) while keeping the |
| | | | | objective of destroying and/or |
| | | | | repelling (depending on use) pests |
| | | | | without harming nature. |
| Deodorants - | X | | X | The capsule must release the active |
| disinfectants. | | | | agent which takes the form of a |
| Anti-moss | | | | fragrance. The capsule must be |
| and anti- | | | | hermetic and function in the form |
| fungal oil | | | | of a nano spray diffusing this |
| | | | | fragrance from a mixture of |
| | | | | essential oils whose active agents, |
| | | | | in the form of gas, will neutralize |
| | | | | moss and fungi, for example in |
| | | | | works of art. The specifications |
| | | | | for such a product provide for a |
| | | | | duration of action of more than one |
| | | | | year. |
| Cosmetic | | | X | The active solution (mixture of |
| textiles: | | | | essential oils) in the capsule must |
| Anti- | | | | be diffused by osmotic pressure |
| microbial, | | | | (nano spray) on the carrier textile |
| anti- | | | | and thus create a protective shell |
| bacteria, | | | | against certain bacteria, viruses |
| anti-virus | | | | and microbes. Double |
| | | | | specifications: A specification |
| | | | | “effectiveness against pathogens” |
| | | | | and a specification on the grip |
| | | | | (the number of potential grips |
| | | | | after washing) of each capsule. |
| Cosmetic | | | X | The active solution included in the |
| textiles. | | | | capsule must be diffused by osmotic |
| Anti | | | | pressure (nano spray) on the |
| cellulite. | | | | carrier textile and create an |
| | | | | action on the pores of the skin |
| | | | | without any liquid coming into |
| | | | | contact with the skin. Double |
| | | | | specifications: An “efficiency” |
| | | | | specification, i.e. that the nano |
| | | | | spray touches the receptors of the |
| | | | | skin to start the process of weight |
| | | | | loss (due to the active agent (eg |
| | | | | coffee)) and specifications on the |
| | | | | hook (in number of potential hooks |
| | | | | after washing) of each capsule. |
| Active | | | X | The capsule must protect its |
| thermal | | | | content from any direct interaction |
| control. | | | | with the outside world, which |
| Phase | | | | allows a smooth transition of |
| change | | | | temperature transition between two |
| materials. | | | | environments separated by this type |
| | | | | of capsule (avoiding rise or fall |
| | | | | of temperature). |
| Photochromic | | | X | The capsule must protect its |
| materials, | | | | contents from any direct |
| reversible | | | | interaction with the outside world, |
| and non- | | | | which allows a change of color at a |
| reversible. | | | | temperature threshold chosen by |
| | | | | specifications. Perfect sealing |
| | | | | required. Protection over time and |
| | | | | other parameters adaptable |
| | | | | according to the specifications. |
| Cosmetics: | | | X | The capsule must keep an active |
| Protection | | | | ingredient intact without leakage. |
| of active | | | | The latter can be released to |
| ingredient. | | | | deploy its function, on the |
| | | | | intervention of an external |
| | | | | “signal” acting on the wall of the |
| | | | | capsule. |
| Cosmetics: | X | | X | The capsule must deliver at a given |
| Release of | | | | time, over a chosen duration and on |
| active | | | | a given area, a liquid active |
| ingredient. | | | | element that provides added value |
| | | | | in terms of efficacy and safety. |
| Cosmetics: | | | X | The capsule must protect its |
| UV filter | | | | contents from any direct |
| | | | | interaction with the outside world. |
| | | | | The content whose purpose is a |
| | | | | “shell against UV” must be able to |
| | | | | last a long time (Specifications). |
| Cosmetics: | | | X | The capsule must protect its |
| Protection | | | | contents from any direct |
| of pigments/ | | | | interaction with the outside world. |
| dyes | | | | The content whose purpose is to |
| | | | | react according to determined |
| | | | | criteria, must be able to last a |
| | | | | long time (Specifications). By |
| | | | | determined criteria, we mean, for |
| | | | | example, changes in color or |
| | | | | intensity depending on the |
| | | | | specifications requested. |
| Cosmetics, | X | X | | The capsule must be able to cross |
| cargo | | | | different parts or zones of the |
| transport | | | | human body (with aggressive |
| for | | | | environments) or be transported to |
| controlled | | | | previously targeted zones, to |
| and | | | | deliver its content at the right |
| targeted | | | | time, in the right place and at the |
| release | | | | right dose (controlled release). |
| Pharmacy. | | X | | The capsule must be able to pass |
| Controlled | | | | through different parts or zones of |
| release of | | | | the human body (with aggressive |
| active and | | | | media) or be transported by blood |
| targeted | | | | or others, to previously targeted |
| ingredients, | | | | areas to deliver its contents, at |
| gastro- | | | | the right time, in the right place |
| resistant. | | | | and at the right dose (controlled |
| Decrease in | | | | release). |
| dissolution | | | | |
| kinetics | | | | |
| Pharmacy. | | X | | The capsule must be able to pass |
| Cargo | | | | through different parts or areas of |
| transport | | | | the human body (with aggressive |
| of active | | | | media) or be transported by blood |
| ingredient | | | | or others, to previously targeted areas |
| with | | | | to deliver its contents, at |
| controlled | | | | the right time, in the right place |
| release | | | | and at the right dose (controlled |
| and target | | | | release). |
| on an organ | | | | |
| through | | | | |
| the blood. | | | | |
| Agri-food. | X | | | The capsule is associated with |
| Taste | | | | foodstuff; it contains an aroma |
| masking | | | | which must be released according to |
| (animal | | | | specifications (temperature, |
| and human | | | | grinding by the teeth, etc.). But |
| food) | | | | as long as they are not solicited, |
| | | | | these capsules must remain neutral. |
| Paper. | X | | | The capsule contains an ink-type |
| Self- | | | | element, and is deposited between |
| copying | | | | two sheets of paper in the form of |
| paper. | | | | a complete nano carpet. Under the |
| | | | | pressure of a pen on sheet 1, the |
| | | | | corresponding capsule on sheet 2 |
| | | | | bursts and a nanomark is printed on |
| | | | | sheet 2. The capsule must be able |
| | | | | to withstand time and a given |
| | | | | pressure before fulfilling its |
| | | | | function. |
| Paper, | X | | | The capsule contains a fragrance or |
| printing, | | | | other active agent. The capsules |
| packaging | | | | are mixed with printing inks or |
| and point- | | | | varnishes. The goal is to create a |
| of-sale | | | | promotional tool to help with sales |
| advertising | | | | releasing, for example, a perfume, |
| | | | | for packaging intended for the |
| | | | | world of perfumery, or even a smell |
| | | | | of a “taste” for the food world |
| | | | | (smell of “bacon chips” for |
| | | | | example). |
| Adhesives | X | | | The capsule must break under a |
| | | | | certain pressure to release a |
| | | | | tackifier to join surfaces. They |
| | | | | can remain inert before use but |
| | | | | must be effective as soon as they |
| | | | | are called upon. |
| Lubricants | X | | | The capsule must break under a |
| and anti | | | | certain pressure to release a |
| friction | | | | lubricant. May remain inert before |
| | | | | use but must be effective as soon |
| | | | | as they are called upon. |
| Welfare. | X | | | The capsule contains either a |
| Deodorants. | | | | perfume or an active agent intended |
| Sjower gel | | | | to be released in liquid form. They |
| and bath | | | | may also contain fragrances and are |
| gel. | | | | then intended to be mixed with |
| Shampo. | | | | neutral or cleansing agents for |
| Toothpaste. | | | | deposition on the skin. Their |
| Cream for | | | | specifications take into account |
| body and | | | | the regulatory constraints of the |
| face etc. | | | | final medium (e.g. the skin) but |
| | | | | also the pH and other factors as |
| | | | | well as the lifespan of the |
| | | | | effectiveness of the final product. |
| Bedding | | | X | The capsule must release the active |
| and | | | | agent in a fragrance form. The |
| equipment. | | | | capsule must be hermetic and |
| Mattresses, | | | | function in the form of a nano |
| pillows, | | | | spray diffusing this fragrance from |
| fabrics, | | | | a mixture of essential oils whose |
| luggage, | | | | active agents, in the form of gas, |
| personal | | | | will neutralize fungi, bacteria and |
| and | | | | others. The specifications for such |
| household | | | | a product provide for a duration of |
| equipment | | | | action of more than one year. |
| Automobiles. | | | X | The capsule must have a long shelf |
| Automotive | | | | life. It must be fixed (or mixed) |
| equipment | | | | on or in various supports |
| and | | | | (textiles, plastics, leather, etc.) |
| accessories | | | | and allow the long-term release of |
| | | | | either an active agent or an active |
| | | | | fragrance, or else protect a heat- |
| | | | | colouring agent. |
| Paintings | | | X | The capsule must have a long shelf |
| with added | | | | life. It must be fixed (or be |
| values | | | | mixed) on or in various supports |
| | | | | (wood, plaster, steel, etc.) and |
| | | | | allow the release over a long |
| | | | | period of time of either an active |
| | | | | agent or an active fragrance, or |
| | | | | even protect a thermocoloring |
| | | | | agent. Creation of value-added |
| | | | | paint ranges. |
| Phytosanitary | X | | X | The capsule must release the active |
| | | | | agent in liquid form over a given |
| | | | | time (X days to X months) or must |
| | | | | deliver the active fragrance |
| | | | | without breaking the shell of the |
| | | | | capsule. The release and/or |
| | | | | delivery time of the active agent |
| | | | | depends on the specification. The |
| | | | | latter recommends a duration, but |
| | | | | must also take into account the |
| | | | | ambient environment (pH, humidity, |
| | | | | temperature, altitude + other |
| | | | | factors (seasons, latitude and |
| | | | | longitude)). |
| Veterinary | X | | | The capsule must be able to pass |
| | | | | through different parts or areas of |
| | | | | the animal body (with aggressive |
| | | | | media) or be transported by blood |
| | | | | or others, to previously targeted areas |
| | | | | to deliver its contents, at |
| | | | | the right time, in the right place |
| | | | | and at the right dose (controlled |
| | | | | release). |
| | | | | The capsule is also designed to be |
| | | | | deposited in the form of a cream, |
| | | | | spray or liquid for curative and |
| | | | | preventive treatments on the skin |
| | | | | of animals. |
| Depilatory | | | X | The capsule must have a |
| cream | | | | thermochromic profile programmed to |
| | | | | change color at a pre-determined |
| | | | | temperature (according to |
| | | | | specifications). These capsules are |
| | | | | designed to indicate the optimal |
| | | | | temperature to deposit a depilatory |
| | | | | cream, without presenting a danger. |
| | | | | These capsules are designed to |
| | | | | withstand high temperatures and |
| | | | | pressures without altering the |
| | | | | indicator function. |

We describe some examples of use in great detail below.

### Example 30: Use of PBAE Microcapsules Containing a Perfume in a Fabric Softener

This test relates to the use of PBAE microcapsules according to the invention as a perfume carrier in a liquid fabric softener product.

Microcapsules according to the invention containing a perfume were prepared, which were separated into three batches:

Batch no. 1: untreated microcapsules;

Batch no. 2: microcapsules reinforced with an “over-coating” process;

Batch no. 3: microcapsules reinforced with a “post-curing” process.

For each batch of microcapsules, 0.3 wt.-% of a slurry of these microcapsules was incorporated into a standard base dose of fabric softener; this base did not contain any perfume (so-called neutral base). Three batches of fabric softener were obtained thereby.

Fifteen cotton napkins were provided (each forming a square of about 32 cm).

For each batch of fabric softener, five napkins were washed in a standard washing machine with a short wash cycle, after adding a standard dose of a standard detergent and a dose of this fabric softener containing the slurry.

The napkins were wrought at 1,000 rpm, put on a line and left to dry for a day. Then they were “caressed” as if it was intended to store them flat. The intensity of the odor was evaluated with a panel of five people, on a scale from 0 (“does not smell”) to 5 (“smells strongly”).

The results are summarized in Table 4 below.

TABLE 4

| | Opening | Napkins | | |
|---|---|---|---|---|
| | of the | put on | Dry | Napkins |
| | door | a line | napkins | « caressed » |
| Batch 1: untreated | 3 | 2 | 1 | 0 |
| Batch 2: reninforced by | 2 | 1 | 1 | 3 |
| « over-coating » | | | | |
| Batch 3: reinforced by | 2 | 1 | 1 | 2 |
| « post curing » | | | | |

Example 31: Use of PBAE microcapsules containing phytosanitary products

This test relates to the use of PBAE microcapsules according to the invention as an essential oil carrier in a composition intended to be applied to cherries.

This use aims at controlling the cherry fly, Rhagoletis cerasi. It is a diptera which measures 4 to 5 mm in length, possessing a single pair of wings. Its body is black, with a yellow patch on its back. Its wings are translucent, streaked with black bands. It only attacks cherries: in spring, from the end of April until June, the adult females land on the fruits, pierce the epidermis and lay their eggs there. A fly can lay between 60 and 70 eggs, with only one egg per cherry. The larvae hatch in one to two weeks, and usually settle in the center of the fruit, around the stone. The maggots thrive by consuming the fruit over three weeks to a month. The affected fruits have a small hole, and brown spots appear.

According to the state of the art, cherries are sprayed with a natural pyrethrum-based insecticide (from Tanacetum cinerariifolium), but its effectiveness remains limited, and will disappear after a few days, due to the oxidation of the active ingredients and their weathering in contact with moisture.

The test was done with two cherry branches, identically exposed, with similar fruit density. On the fruits of branch A, a cloud of pyrethrum aerosol was sprayed. On the fruits of branch B, a cloud of an aerosol of “unbreakable” type PBAE microcapsules containing pyrethrum was sprayed.

On branch A, after 12 to 14 days, a small quantity of fruits are observed which are pierced; a week later their number is greater; contaminated fruit contains a maggot. After 25 days, a very large majority of cherries are holed and all harbor one or more worms. Over the same period, on branch B, we observe: after 14 days there are no cherries with holes, after 21 days three cherries with holes (under the leaves closest to the ground), after 25 days two other cherries with holes, near the first three. This may be related to poor manual spreading of the aerosol.

It is concluded that the use of the product according to the invention has a significantly better efficacy. This is due to the better protection of the active product against oxidation and moisture.

### Example 32: Use of PBAE Microcapsules in Deodorant Compositions

This test aims to compare a product A, containing microcapsules of the polyurea type containing a deodorant, with a product B, containing PBAE microcapsules of the “fragile” type containing the same deodorant.

A composition comprising product A and a composition comprising product B were prepared. Using a sponge, 0.40 g of each composition was spread on a nonwoven fabric of dimensions 12 cm×10 cm.

These tissues were used with a panel of people accustomed to using deodorants to assess the smell, one day after washing the skin, before and after rubbing with the fabric. A rating of 1 to 10 was used (10 being the best rating).

The following results were obtained:

Product A (polyurea): at rest=score 4 out of 10; after rubbing=score 8 out of 10

Product B(PBAE): at rest=score 3 out of 10; after rubbing=score 10 out of 10.

This difference is commercially significant.

### Example 33: Use of PBAE Microcapsules in Biocidal Compositions

This test aims to compare the action of a product A (composition of pure essential oils) applied in the form of an aerosol, with the action of a product B (60% water, 40% microcapsules according to the invention containing the same composition as product A) applied in the form of an aerosol. The action was evaluated against bedbugs, a species of heteroptera insects of the Cimicidae family. The insects came from a farm, are all of an identical stage of development, and were fed on human blood using an artificial membrane and a Hemotek device. After gorging, they were placed in two Petri dishes with a diameter of 90 mm closed by a fabric cover, at the rate of 15 insects per box. Beforehand, the products A or B were spread on a blotting paper placed at the bottom of the box, and left to dry.

The number of dead insects was then recorded after each day, for eight days.

The results are collated in Table 5:

| J | 1 | 2 | 3 | 4 | 5 | 6 | 7 | 8 | Survivors after 8 days |
|---|---|---|---|---|---|---|---|---|---|
| Box A | 5 | 2 | | | | | | 1 | 7 |
| Box B | 3 | 3 | 3 | 2 | 4 | | | | 0 |

It is observed that product B (non-encapsulated) loses its effectiveness after two days, whereas the encapsulated product has an effectiveness which is spread over at least five days (end of the test after having killed all the insects).

1. A composition comprising microcapsules with a biodegradable shell made of Poly(Beta-Amino Ester), abbreviated as PBAE, containing at least one active substance, and formulated as a product selected from the group consisting of:
Cosmetic products and care products intended to be applied to the face or on the body, in particular creams and lotions for the body, creams and lotions for the face, shower gels, bath gels, products with UV filters, self-adaptive make-up products correcting their shade by function of solar or electric ambient lighting, said at least one microencapsulated active substance being in particular intended to be released in a controlled and targeted manner, in particular for vectorization in the skin, for stabilization of pigments and/or dyes, for covering of wrinkles;
Hair and scalp care products, including shampoos, conditioners, hair dyes;
Depilatory waxes, said at least one microencapsulated active substance being preferably selected from the group formed by thermochromic substances and agents for temperature control;
Disinfectant and/or deodorant products intended to be applied to the face or body, or to objects in contact with the body, to control or limit or mask body odor or to control or limit or mask body odor objects in contact with the body, said at least one active substance being preferably selected from the group formed by antiperspirants, perfumes, essential oils, fragrances, oils;
Cosmetic textiles, intended to come into contact with the skin, said at least one active substance preferably being selected from the group formed by antioxidant products, anti-cellulite products, products capable of stimulating blood circulation, anti-mold products, antimicrobial products, bactericidal products, virucidal products, oils;
Pharmaceutical preparations, in particular intended for use orally, nasally, intraveneously, rectally or by topical application, for example cutaneous or ocular, said at least one active substance preferably being selected from the pharmaceutical active ingredients;
Preparations for the care of the teeth and the mouth, said at least one active substance being preferably selected from bactericidal products, enzymes, flavorings, essential oils, sweeteners;
Food preparations, said at least one active substance being preferably selected from flavorings, flavor enhancers, vitamins and vitamin preparations, preservatives, nutritional additives, oils;
Insecticidal and/or fungicidal and/or repellent and/or anti-moss preparations, intended for use alone or incorporated into products such as phytosanitary products, paints, coatings, textiles, detergents, plastics;
Preparations intended for use in the field of agriculture, said at least one active substance being preferably selected from additives for the cultivation of plants and fertilizers;
Preparations intended for veterinary, intracorporeal or extracorporeal use, and in particular pharmaceutical preparations, dermatological preparations and coat care preparations, said at least one active substance being preferably selected from pharmaceutical active ingredients and in particular dewormers and antiparasitics, food additives, perfumes, fragrances;
Preparations for the treatment of textile products, textile fibers or footwear, said at least one active substance preferably being selected from biocidal products, repellents, moth repellents, deodorizing products, self-cleaning products, stain-resistant products, antistatic products, self-healing products, self-repairing products, perfumes, fragrances, detergents, softeners;
Preparations for the treatment of bedding, in particular mattresses, pillows, sheets, draw sheets, luggage, fabrics and household equipment such as curtains, cushions, furniture, leather objects, said at least one active substance being preference selected from among biocidal products, repellents, moth repellents, anti-mite products, anti-lice products, anti-mosquito products, deodorant products, self-cleaning products, antistatic products, self-healing products, self-repairing products, perfumes, fragrances, detergents, softeners;
Preparations for waterproof or water-repellent coatings, intended to be applied in particular to textile surfaces or leather surfaces, said at least one active substance preferably being selected from hydrophobic compounds, in particular fatty compounds or fluorinated compounds;
Deodorant and/or disinfectant and/or antifoam and/or antifungal preparations, said at least one active substance preferably being selected from oily, natural or synthetic products;
Detergent products;
Glues and adhesives;
Preparations for coating building materials,
preparations for ensuring watertightness and preparations for thermal insulation or for absorbing and restoring thermal energy, said at least one active substance preferably being selected from phase, paints and varnishes, thermal insulation materials, sealing agents, expansion agents;
Products for treating floor coverings, said at least one active substance being preferably selected from the group formed by lubricant products, anti-friction products, anti-slip products, detergents, essential oils, fragrances, perfumes, aromas;
Paints, varnishes, dyes and inks, in particular inks for transfer printing, screen printing, ink jet printing or electrostatic printing, and preparations for coating or making paper, said at least one active substance preferably being selected from the group formed by dyes, pigments, thermochromic products, photochromic products, perfumes, flavorings, fragrances;
Self-repairing compositions intended in particular for use in surface coatings used in paint, varnish, inks, on cement, concrete, wood, and in compositions of polymer materials and composites;
Compositions with a fire retardant and fire extinguisher effect, said at least one active substance being able in particular to be chosen from brominated alkanes of general formula CₙH₂ₙ₊₂₋ₓBrₓ.

2. The composition according to claim 1, wherein said microcapsules have been obtained by a process in which:
(a) an aqueous solution of a surfactant, an oily phase comprising said active substance and at least one first monomer X, and a polar phase comprising at least one second monomer Y are provided;
(b) an emulsion of the O/W type is prepared by adding said oily phase to said aqueous solution of the surfactant;
(c) said polar phase is added to said O/W emulsion, to enable a polymer to be obtained by polymerization of said monomers X and Y, said polymer forming the shell of said microcapsules;
and in which said first monomer X is preferably selected from (multi)acrylates, and preferably (multi)acrylates of formula X′—(—O(C═O)—CH═CH₂)ₙ with n>4 and where X′ represents a molecule on which n acrylate units are grafted, or selected from the group formed by:
diacrylates;
triacrylates, in particular trimethylolpropane triacrylate, tetraacrylates, pentaacrylates, hexaacrylates, mixtures of these different acrylates of the O[CH₂C(CH₂OR)₃]2 type where R is H or COCH═CH₂;
polymers bearing pendant acrylate functions;
functional oligo PBAEs, prepared for example by reacting diacrylate compounds with a functional primary amine and/or a functional secondary diamine;
the mixture of different compounds described above.

3. The composition according to claim 1, wherein the said second monomer Y is selected from amines and preferably selected from the group formed by:
primary amines R—NH₂;
primary diamines of the NH₂(CH₂),NH₂ type, where n is an integer which can typically be between 1 and 20, and which is preferably 2 or 6;
primary diamines having an aromatic core, and preferably meta-xylylene diamine;
(multi)primary amines, and preferably tris(2-aminoethyl)amine;
(multi)amines containing primary and secondary amine functions, and preferably tetraethylene pentamine;
secondary diamines and preferably piperazine;
polymers containing primary and or secondary amine functions, and preferably polyethylene imine.

4. The composition according to claim 2, wherein the ratio of the reactive functions of the said monomers Y (—NH) and X (acrylate) is greater than 1, preferably between 1 and 5, and even more preferably between 1.2 and 3.8.

5. The composition according to claim 2, wherein the said polymerization of the said monomers is carried out under stirring at a temperature of between 20° C. and 100° C., and preferably between 30° C. and 90° C.

6. The composition according to claim 1, wherein the external shell of the microcapsules is modified by one of the following ways:
Deposition of a coating on the surface of the microcapsule, preferably from a polymer dispersed in an aqueous phase, said polymer preferably being selected from polysaccharides, such as cellulose, starch, alginates, chitosan, and their derivatives;
Addition of a radical initiator in the aqueous phase and/or the oily phase, said radical initiator preferably being selected:
If added in the aqueous phase: from the group formed by water-soluble azo compounds, such as 2,2′-Azobis(2-methylpropionamidine) dihydrochloride, and red-ox systems, such as ammonium persulphate or potassium persulphate in combination with potassium metabisulphite;
If added in the oily phase: from the group formed by azo compounds, such as azobis-isobutyronitrile and its derivatives, and peroxidic compounds, such as lauroyl peroxide;
Addition in the aqueous phase of a water-soluble acrylate capable of modifying the surface state of the microcapsules, said acrylate preferably being a water-soluble monofunctional acrylate capable of reacting with the residual amine functions at the surface of the shell of the microcapsules, said water-soluble monofunctional acrylates being preferably selected from the group formed by 2-carboxyethyl acrylate, 2-(dimethylamino)ethyl acrylate, 2-hydroxyethyl acrylate, poly(ethylene glycol) acrylates, the potassium salt of 3-sulfopropyl acrylate.

7. The composition according to claim 1, wherein the said microcapsules are characterized by one of the following characteristics or combination of characteristics:
an indentation force at break greater than 5,000 μN, preferably greater than 6,000 μN, and even more preferably greater than 7,000 μN; these microcapsules being referred to herein as “unbreakable”;
an indentation force at break of less than 300 μN and preferably a Tg value of less than 33° C., and in this case preferably less than 32° C. and even more preferably less than 31° C.; preferably by an indentation force at break of less than 250 μN and preferably by a Tg value of less than 32° C., and in this case preferably less than 31° C. and even more preferably less than 30° C.; and even more preferably by an indentation force at break of less than 200 μN and preferably by a Tg value of less than 32° C., and in this case preferably less than 31° C. and even more preferably less than 30° C.; these microcapsules being referred to herein as “fragile”;
an indentation force at break between 200 μN and 5,000 μN, and preferably between 300 μN and 5,000 μN, but with a breaking force between 200 μN and 300 μN their Tg value is preferably at least 33° C., and for the microcapsules with a breaking force greater than 300 μN it is preferable that their Tg value be between 30° C., preferably between 33° C., and 80° C.; these microcapsules being referred to herein as “brittle”.

8. The composition according to claim 7, wherein unbreakable microcapsules are used for the formulation of a product selected from the group consisting of:
preparations for thermal insulation or for absorption and restitution of thermal energy, and preferably such preparations comprising microcapsules containing phase change materials;
preparations comprising microcapsules containing a thermochromic or photochromic substance, intended in particular to be used in depilatory waxes, packaging of food products to be heated, labels for refrigerated products making it possible to indicate a possible break in the cold chain, coatings allowing the indication of excessive stress or impacts following an accident, wear or misuse, in particular on plastic or composite parts, on mountaineering ropes, technical fabrics or protective helmets.

9. The composition according to claim 7, wherein fragile microcapsules are used for the formulation of a product selected from the group consisting of:
compositions for the treatment of the hair and the scalp,
cosmetic preparations and care products, and preferably such preparations comprising microcapsules containing pigments or dyes;
food preparations, and preferably such preparations comprising microcapsules containing active food products, such as flavorings, flavor enhancers, vitamins, nutritional additives, preservatives;
phytosanitary products, paints, coatings, textile products, detergents, plastic materials, products for the surface treatment of wood, and preferably such products comprising microcapsules containing insecticides, repellents, fungicides, anti-moss products, termite treatment products;
preparations for agriculture, and preferably such preparations comprising microcapsules containing additives for the cultivation of plants, and/or fertilizers;
phytosanitary preparations;
pharmaceutical, food or care preparations intended for use in the veterinary field, and preferably such preparations comprising microcapsules containing pharmaceutical or dermatological active ingredients or active ingredients for the care of the coat, such as vermifuges, anti-parasites or agents for controlling or limiting odors;
compositions for waterproof or water-repellent coatings, in particular intended to be applied to textile surfaces or leather surfaces, and preferably such preparations comprising microcapsules containing hydrophobic compounds, in particular fatty compounds or fluorinated compounds.

10. The composition according to claim 7, wherein brittle microcapsules are used for the formulation of a product selected from the group consisting of:
products in which it is desired to be able to cause the release of the active substance by the application of mechanical pressure or by a chemical modification of the microcapsule which modifies its mechanical properties;
preparations for blowing agents, sealants, adhesives or glues;
cosmetic and/or dermatological preparations, care products, in particular shower and bath gel products, deodorants and products for odor control, and preferably such preparations comprising microcapsules containing dermatological or cosmetic active ingredients;
preparations for coating cosmetic textiles, and preferably such preparations comprising microcapsules containing antioxidant products, anti-cellulite products, products capable of stimulating blood circulation, as well as anti-mould, anti-microbial, bactericidal and virucidal products;
preparations for treating or coating textile products or textile fibers, in particular technical products and fibers, and preferably such preparations comprising microcapsules containing detergents, softeners, biocides, antistatic products, anti-stain products;
preparations for coating, treatment or maintenance of floors, and preferably such preparations comprising microcapsules containing lubricants and/or anti-friction products, or anti-slip products, or containing essential oils, fragrances, perfumes, and/or flavorings;
preparations having a fire retardant and extinguisher function, and preferably such preparations comprising microcapsules containing brominated alkanes of general formula CₙH₂ₙ₊₂₋ₓBrₓ;
preparations for self-repair, in particular for self-repair following an impact, and preferably such preparations comprising microcapsules containing self-repair products;
preparations for coating paper or for incorporation into a preparation for making paper, and in particular scented inks of the scratch & sniff or rub & sniff type, and preferably such preparations comprising microcapsules containing perfumes, aromas, fragrances, thermochromic substances or adhesives;
preparations intended for use in the veterinary field for care products, pharmaceutical compositions and food products, and preferably such preparations comprising microcapsules containing products selected from the group formed by vermifuges, anti-parasites, agents for controlling or limiting odors, pharmaceutically active substances, essential oils, fragrances, perfumes, and/or flavorings, which can be used in many products, by mixing into the product or by applying a coating on the product;
preparations intended for the field of agriculture; and preferably such preparations comprising microcapsules containing additives for the cultivation of plants and/or fertilizers;
preparations for the treatment of bedding, luggage, fabrics and household equipment, in particular curtains, furniture with fabrics, furniture and other leather objects, cushions, and preferably such preparations comprising microcapsules containing biocidal products and/or repellent products, and in particular anti-mite products, anti-lice products, anti-moth products, anti-mosquito products, and/or anti-stain products, perfumes, essential oils, fragrances and/or flavorings;
preparations ensuring watertightness, to be applied to solid materials, for example in the building sector, or flexible, and preferably such preparations comprising microcapsules containing sealing agents and/or expansion agents.

11. The composition according to claim 2, wherein the reaction mixture resulting from stage (c) is used directly, optionally after adjustment of the pH value.

12. The composition according to claim 6, wherein the reaction mixture resulting from the said modification reaction is used directly.

13. The composition according to claim 2, wherein the microcapsules are used after washing and optionally drying.

14. The composition according to claim 13, in which the washed microcapsules are used for the formulation of a product selected from the group formed by: pharmaceutical preparations intended for human or veterinary medicine, food preparations, cosmetic products, products for dental and oral care, preparations of additives for plant cultivation, preparations for coating cosmetic textiles or medical textiles.

15. A method of preparing the composition of claim 1.

16. Method according to claim 15, comprising a step in which the microcapsules, preferably in the form of slurry, are incorporated into a liquid, viscous or pasty preparation, said preparation being intended to be used, or used in a subsequent step, as such, so as to enable the microcapsules to fulfil a desired technical function, and/or said preparation being used as a vehicle for applying the microcapsules to another product, in particular to a solid product, to deploy therein a sought-after technical function.

17. Product resulting from the use or from a method of use, of microcapsules with a biodegradable Poly(Beta-Amino Ester) shell containing at least one active substance, according to claim 1.

18. The composition according to claim 7, wherein the reaction mixture resulting from stage (c) is used directly, optionally after adjustment of the pH value.
